(19) United States
(12) Patent Application Publication
(10) Pub. No.: US 2023/0384306 A1
(43) Pub. Date: Nov. 30, 2023

(54) AUTOANTIBODIES, KITS AND METHODS OF VERIFYING THE ACCURACY OF DIAGNOSTIC RESULTS

(71) Applicant: Arizona Board of Regents on Behalf of Arizona State University, Scottsdale, AZ (US)

(72) Inventors: Mahasish SHOME, Tempe, AZ (US); Yunro CHUNG, Chandler, AZ (US); Ramani CHAVAN, Tempe, AZ (US); Jin PARK, Phoenix, AZ (US); Ji QIU, Chandler, AZ (US); Joshua LABAER, Chandler, AZ (US)

(21) Appl. No.: 18/326,837

(22) Filed: May 31, 2023

Related U.S. Application Data
(60) Provisional application No. 63/347,560, filed on May 31, 2022.

(51) Int. Cl. G01N 33/564; G01N 33/569; G01N 33/574

(52) U.S. Cl. CPC G01N 33/564; G01N 33/56983; G01N 33/57484

(57) ABSTRACT

The present invention relates autoantibody biomarkers, kits, and methods for increasing the accuracy of a cancer or autoimmune screening of a subject suspected of having cancer or an autoimmune condition.

### CROSS-REFERENCE TO RELATED APPLICATIONS

This application claims the benefit of and priority to U.S. provisional patent application 63/347,560, filed May 31, 2022, the entirety of the disclosure of which is hereby incorporated by this reference thereto.

### STATEMENT REGARDING FEDERALLY SPONSORED RESEARCH OR DEVELOPMENT

This invention was made with government support under U01 CA214201 awarded by the National Institutes of Health. The government has certain rights in the invention.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 29,679 byte XML file named “SeqList” created on May 23, 2023.

### FIELD OF THE INVENTION

The invention relates to autoantibodies, methods, kits and their use to identify false positive antibody markers of cancer or autoimmune condition.

### BACKGROUND OF THE INVENTION

Autoantibodies have been reported in individuals with autoimmune disease and cancer. They were believed to be absent in healthy individuals due to the immune tolerance mechanism; however, some have been found frequently in healthy individuals, and these antibodies are called common autoantibodies. The presence of common autoantibodies can confound the search for disease-linked autoantibodies, and their documentation will simplify the identification of autoantibodies specific to certain diseases. Indeed, only a small fraction of the autoantibodies reported in the literature have been validated in independent cohorts, suggesting the classification performance for many reported autoantibodies requires further investigation.

A comprehensive documentation of common autoantibodies will facilitate the elucidation of the complex immunology underlying their elicitation. One class of common autoantibodies is referred to as natural antibodies (NAbs). Unlike adaptive antibodies, NAbs are synthesized by B1 lymphocytes (bearing CD20⁺CD27⁺CD43⁺CD70⁻) and marginal zone B cells, and do not undergo affinity maturation by antigen stimulation or extensive somatic mutation (Coutinho et al., 1995). Another class of common autoantibodies may arise from cross-reactive antibodies to infectious agent proteins when the similarity in foreign and self-peptides may activate self-reactive T or B cells. It has been experimentally demonstrated that patients with either measles virus or herpes simplex virus type 1 produce antibodies against a viral phosphoprotein that cross-react with an intermediate filament protein of human cells. Additionally, transgenic mice infected with lymphocytic choriomeningitis virus (LCMV) may develop chronic inflammation in the central nervous system (CNS) due to epitopes shared between LCMV proteins and CNS antigens. Several bioinformatics techniques have been developed to discover potential mimicry candidates.

The immunogenicity of a protein can be attributed to its intrinsic properties and extrinsic responses by the host. Biochemical and structural properties like flexibility, hydrophilicity and beta-turns can promote antigenicity while hydrophobicity, alpha-helices and beta-sheets can suppress antigenicity. That these common autoantibodies do not cause evident autoimmune disease is intriguing. The presence of autoantibodies in serum reflects leakiness of central and/or peripheral tolerance mechanisms. However, their presence does not guarantee a causal role in autoimmune disease development. For autoantibody-induced pathology, the autoantibody needs to bind to the autoantigen to form an immune complex. Sequestration of the autoantigen from autoantibodies can inhibit the autoantibody-induced pathology.

### SUMMARY OF THE INVENTION

Described herein is a method of increasing the accuracy of a cancer or an autoimmune condition screening in a subject suspected of having cancer or an autoimmune condition by identifying at least one false positive indicator. The method comprises providing a biological sample from the subject; detecting the presence of one or more autoantibodies against at least one autoantigen listed in Table 1 in the biological sample and identifying at least one false positive indicator upon detecting the presence of the one or more autoantibodies. Also described is a method of verifying the accuracy of a diagnosis of a cancer or autoimmune condition in a subject. The method comprises providing a biological sample from the subject; detecting the presence of one or more autoantibodies against at least one autoantigen listed in Table 1 in the biological sample, an identifying the subject as falsely diagnosed with cancer or an autoimmune condition upon detecting the presence of the one or more autoantibodies.

In some implementations, the at least one autoantigen is selected from the group consisting of: AMY2A, SPAG8, GTSE1, MAK, RNF138, PMFBP1, CCDC34, ODF2, CSF3, CAPN3, MYLK2, TRIM29, SOX2, and STMN4. Where the biological sample is from the subject's pancreas, the at least one autoantigen is AMY2A. Where the biological sample is from the subject's lungs, the at least one autoantigen is CSF3. Where the biological sample is from the subject's salivary gland, nerve tibial, or brain, the at least one autoantigen is SOX2. Where the biological sample is from the subject's esophagus, skin, or vagina, the at least one autoantigen is TRIM29. Where the biological sample is from the subject's skeletal muscle, the at least one autoantigen is CAPN3 and/or MYLK2. Where the biological sample is from the subject's testis, the at least one autoantigen is selected from the group consisting of: SPAG8, GTSE1, MAK, RNF138, PMFBP1, CCDC34, and ODF2.

In other implementations, the at least one autoantigen is selected from the group consisting of: STMN4, ODF4, RBPJ, AMY2A, EPCAM, ZNF688, CSF3, RAD51AP1, PSKH1, LENG1, S1PR3, LYSMD1, FAM76A, CDR2L, and CCDC130. In some embodiments, where the at least one autoantigen is EPCAM, the method further comprises detecting the presence of at least one other autoantibody against EDG3 or CSF3. Detecting the presence of the at least one other autoantibody against EDG3 or CSF3 in addition to the presence of at least one autoantibody against EPCAM in the biological sample indicates that the subject is false positive for having cancer or an autoimmune condition.

In some aspects, the method comprises detecting at least two, at least three, at least four, or at least five autoantibodies against at least two, at least three, at least four, or at least five autoantigens selected from the group consisting of: STMN4, ODF4, RBPJ, AMY2A, EPCAM, ZNF688, CSF3, RAD51AP1, PSKH1, LENG1, S1PR3, LYSMD1, FAM76A, CDR2L, and CCDC130. Detecting the presence of the at least two, at least three, at least four, or at least five autoantibodies indicates the subject is false positive for having cancer or an autoimmune condition. In certain embodiments, wherein the method comprises detecting at least ten autoantibodies against at least ten selected from the group consisting of: STMN4, ODF4, RBPJ, AMY2A, EPCAM, ZNF688, CSF3, RAD51AP1, PSKH1, LENG1, S1PR3, LYSMD1, FAM76A, CDR2L, and CCDC130, detecting the presence of the at least ten autoantibodies indicate the subject is false positive for having cancer or an autoimmune condition. In a particular embodiment, the method comprises detecting the presence of at least one autoantibody against STMN4, ODF4, RBPJ, AMY2A, EPCAM, ZNF688, CSF3, RAD51AP1, PSKH1, LENG1, S1PR3, LYSMD1, FAM76A, CDR2L, and CCDC130 in the biological sample. Detecting the presence of at least one autoantibody against STMN4, ODF4, RBPJ, AMY2A, EPCAM, ZNF688, CSF3, RAD51AP1, PSKH1, LENG1, S1PR3, LYSMD1, FAM76A, CDR2L, and CCDC130 in the biological sample indicate the subject is false positive for having cancer or an autoimmune condition.

In certain implementations, the method comprises detecting the presence of at least one autoantibody against at least one autoantigen selected from the group consisting of: STMN4, ODF2, RBPJ, AMY2A, EPCAM, and ZNF688 in the biological sample, wherein the presence of the at least one autoantibody indicates the subject is false positive for having cancer or an autoimmune condition. In some aspects, the method comprises detecting the presence of at least two, at least three, at least four, or at least five autoantibodies against at least two, at least three, at least four, or at least five autoantigens selected from the group consisting of: STMN4, ODF2, RBPJ, AMY2A, EPCAM, and ZNF688 in the biological sample. Detecting the presence of the at least two, at least three, at least four, or at least five autoantibodies indicates the subject is false positive for having cancer or an autoimmune condition. In particular embodiments, the method comprises detecting the presence of at least one autoantibody against STMN4, ODF2, RBPJ, AMY2A, EPCAM, and ZNF688 in the biological sample. Detecting the presence of the at least one autoantibody against STMN4, ODF2, RBPJ, AMY2A, EPCAM, and ZNF688 in the biological sample indicates the subject is false positive for having cancer or an autoimmune condition.

A kit for diagnosing and verifying an autoimmune condition or cancer in a subject is further described. The kit comprises at least one antigen or antibody targeting a cancer marker, an autoimmune marker, or both and a false positive indicator, the false positive indicator comprising at least one autoantigen listed in Table 1. In some embodiments, the kit further comprises at least one labeled secondary antibody or antigen for detecting the binding of the at least one antigen or antibody with the cancer marker and/or the autoimmune marker; and at least one labeled secondary antibody or antigen detecting binding of at least one autoantibody to the false positive indicator. In some aspects, wherein the cancer marker is P53. In other aspects, the autoimmune marker is antinuclear antibodies.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D depict, in accordance with certain embodiments, the development of autoantibody in healthy subjects. FIG. 1A shows the number of antibodies detected in subjects divided into five age groups, based on human development stages. Each gray dot represents the number of autoantibodies found in a healthy subject belonging to that age group. The number of autoantibodies increased significantly over the first two age groups (P<0.001). The horizontal black bar represents median with interquartile range. FIG. 1B is a comparison of number of autoantibodies in female and male healthy subjects. A black dot represents the number of autoantibodies found in a single female subject; while a gray dot, in a single male subject. There were no significant differences between male and female for the number of autoantibodies (two-sample unpaired t-test, P=0.17). The horizontal black bar represents median with interquartile range. FIG. 1C is a comparison of weighted prevalence of common autoantibodies in male and female healthy subjects. A gray bar represents the weighted prevalence of a common autoantibody in the male population, while a black bar below the gray one represents the weighted prevalence of the same autoantibody in the female population. Common autoantibodies are ranked from left to right based on their overall prevalence in healthy subjects. Names of the autoantigens and their ranks are listed in the Table 1. No significant difference between male and female for the weighted prevalence was observed (paired t-test, P=0.06). FIG. 1D depicts the Pearson correlation of common autoantibodies frequency in healthy and diseased cohorts (r=0.975). Each dot represents an autoantigen, against which the autoantibody frequency in either cohort is shown.

FIG. 2 depicts, in accordance with certain embodiments, a schematic diagram for the discovery of 7 or more ungapped amino acids match between common autoantigens and viral proteins.

FIGS. 3A-3G depict, in accordance with certain embodiments, a gene set enrichment analysis (GSEA) of common autoantigens for various biochemical and structural properties. FIGS. 3A-2D show shows primary structure enrichment analysis as labeled. FIGS. 3E-3G show antigenicity and secondary structure prediction method enrichment analysis as labeled. The grey curve on the graph represents the values of the property sorted in descending order for all the proteins studied. The black vertical lines on the graph show where the common autoantigens appear in the ranked list. The curve corresponds to the enrichment score, which is calculated by walking down the ranked list, increasing it when a gene is encountered from the gene set and decreasing it when the encountered gene is not from the gene set. The red color gradient is used to represent positive values while the blue color gradient is used to represent negative values. Concentration of vertical lines on the graph towards a side signifies enrichment while randomly dispersion of vertical lines on the graph signifies no enrichment.

FIGS. 4A-4C are related to FIGS. 3A-3G and depict the Gene Set Enrichment Analysis (GSEA) of common autoantigens for various biochemical and structural properties. FIG. 4A shows the protein length. FIG. 4B show the fraction of amino acids in beta-sheets. FIG. 4C show the surface accessibility prediction. These properties did not lead to significant enrichment. The grey colored curve on the graph represents the values of the property sorted in descending order for all the proteins studied. The black vertical lines on the graph show where the common autoantigens appear in the ranked list. The green curve corresponds to the enrichment score, which is calculated by walking down the ranked list, increasing it when a gene is encountered from the gene set and decreasing it when the encountered gene is not from the gene set. The red color gradient is used to represent positive values while the blue color gradient is used to represent negative values. Concentration of vertical lines on the graph towards a side signifies enrichment while randomly dispersion of vertical lines on the graph signifies no enrichment.

FIGS. 5A and 5B shows the subcellular localization and tissue expression of common autoantigens. FIG. 5A depicts the subcellular localization of all proteins and common autoantigens on the microarrays. FIG. 5B depicts the expression profiles of organ/tissue-specific common autoantigens. Each row represents an organ as labelled on the right and each column represents an autoantigen as labelled at the bottom. Gene expression in transcripts per million (TPM) from GTEx dataset was standardized to the Z scores for data visualization. Organs and autoantigens were clustered based on correlation-based average-linkage clustering.

FIG. 6 is related FIG. 4B and show the expression profiles of common autoantigens in different organs. Each row represents an organ as labelled on the right and each column represents an autoantigen as labelled at the bottom. Gene expression in transcripts per million (TPM) from GTEx dataset was standardized to the Z scores for data visualization. Organs and autoantigens were clustered based on correlation-based average-linkage clustering.

FIG. 7, in accordance with certain embodiments, show the proteins investigated by the 9 studies used for the meta-analysis. All 8282 unique human proteins are represented on the x-axis and the proteins analyzed in each study is shown as a line overlapping with the x-axis corresponding to the labels on the y-axis. There were 123 proteins studied by all studies and 7,242 proteins by studies I, II, III, VIII and IX.

FIGS. 8A and 8B, in accordance with certain embodiments, depict the correlation of co-occurrence of common autoantibodies in healthy and diseased cohorts. Phi correlation coefficient was calculated for each pair of autoantibodies and shown as a heatmap for healthy cohort (FIG. 8A) and diseased cohort (FIG. 8B). The grey color on the heatmaps represent pairs of autoantibodies whose phi correlation coefficient was not defined.

### DETAILED DESCRIPTION OF THE INVENTION

Detailed aspects and applications of the invention are described below in the drawings and detailed description of the invention. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the invention. It will be understood, however, by those skilled in the relevant arts, that the present invention may be practiced without these specific details. It should be noted that there are many different and alternative configurations, devices, and technologies to which the disclosed inventions may be applied. The full scope of the inventions is not limited to the examples that are described below.

The singular forms “a,” “an,” and “the” include plural referents unless the context clearly dictates otherwise. Thus, for example, reference to “a step” includes reference to one or more of such steps.

As used herein, the term “false positive” refers a test result which incorrectly indicates that a particular condition or attribute is present. Accordingly, the “false positive indicator”, in some aspects, refers to a biomarker that indicates the corresponding positive test result incorrectly indicates the presence of a particular condition or attribute, for example, cancer or an autoimmune condition.

Disclosed herein are a selection of common IgG autoantibodies that can be found in healthy individuals and thus are suitable target for identifying false positives in cancer or autoimmune condition diagnostics methods and assays.

Autoantibodies can be broadly divided into two types: 1) pathogenic autoantibodies which contribute to various immune-mediated diseases and 2) common autoantibodies which are found in apparently healthy individuals. While pathogenic autoantibodies can lead to autoimmune diseases, common autoantibodies can bind to a variety of microbial components, thereby providing first line of defense against infections (Elkon & Casali, 2008). They can also recognize self-antigens which help in B cell repertoire development and homeostasis of the immune system. Some of these common autoantibodies occur frequently enough to confound studies intended to find disease-related autoantibodies.

The number of unique IgG autoantibodies in healthy individuals increased with age from infancy to adolescence and then plateaued. This observation suggests that while response to infectious agents (and maybe vaccines) might contribute to autoantibodies through molecular mimicry, this mechanism does not appear to continue to accumulate autoantibodies throughout life. Gender did not appear to play a role in autoantibody production in healthy individuals. This stands in contrast to the observation that autoimmune diseases disproportionally affect females compared to males because male-predominant autoimmune disease is associated with acute inflammation, whereas female-predominant autoimmune disease is associated with antibody-mediated pathology. Several common autoantibodies co-occurred frequently. This could occur if the same antibody recognized two different proteins that share a common epitope. Other possibilities include sharing common HLA haplotypes or playing similar biological roles that lead to escape from tolerance. It is notable that the targets of several of the co-occurring antibodies play roles in stem cell proliferation and differentiation (EPCAM, EDG3 and CSF3) and two others play roles in DNA damage repair (PML and PSMD2). The meaning of this is not clear, but it occurred frequently enough (Phi correlation coefficient >0.6) that it is worth further investigation.

Viral proteins with sequences similar to a human protein may initiate cross-reactive antibodies leading to autoimmunity. There are around 20 autoimmune diseases reported in literature where autoantibodies are generated due to cross-reactivity to infectious agent proteins. Some of the common autoantibodies may be a result from cross-reactivity from anti-viral antibodies, albeit without causing subsequent pathology. The typical length of linear epitope of antibodies ranges from 7 to 9 amino acids and hence these specific matches have the potential to elicit cross-reactive antibodies. The fact that these matches occur significantly more frequently between viral proteins and common autoantigens but less frequently for unreactive proteins on the microarrays further suggests the role of molecular mimicry in common autoantibody elicitation.

The intrinsic properties of a protein, such as its chemical and structural complexity, can impact its antigenicity. Gene Set Enrichment Analysis (GSEA) revealed that common autoantigens tended to favor more basic, hydrophilic with fewer aromatic amino acids. In addition, common autoantigens were also found to be more flexible and have more beta-turns. Flexibility is a property that can help the polypeptide chain to bind easily to immunoglobulin compared to a stiff polypeptide chain. Also, beta-turns can be a potential site for antibody binding as the peptide chain reverses its direction at beta-turns with side chain projected outwards.

Accessibility of autoantigens to circulating autoantibodies is critical to autoimmune disease pathology. In systemic autoimmune diseases, a majority of the target antigens are intracellular molecules and therefore not normally accessible to the B cells or antibodies. Only after excessive cell death or ineffective clearance of apoptotic debris do these intracellular autoantigens become available for immune complex formation. In Wegener's granulomatosis, the autoantigen is an intracellular protease that becomes accessible to the autoantibodies only after an infection triggers translocation of the protease to the surface. Similarly, the autoantigen in Goodpasture's syndrome, normally ensconced in the basal membranes of alveolar capillaries, becomes accessible to the antibodies after an environmental insult to the capillaries, leading to pulmonary hemorrhage. A majority of the common autoantigens identified were located exclusively at intracellular sites, which make them inaccessible to circulating autoantibodies. Some of the common autoantigens are organ/tissue-specific and predominately expressed in the testis and brain, which are isolated from the immune system by the blood-testis or blood-brain barriers, respectively. No obvious form of sequestration was identified for the remaining autoantigens although this cannot be ruled out.

Thousands of studies over the past decade have investigated autoantibodies as potential biomarkers for disease risk assessment, diagnosis, and prognosis. Given the prevalence observed for these common autoantibodies in healthy individuals, in some cases exceeding a quarter of all individuals, they will be frequently encountered in such studies and may confound them as false positives. Table 1 lists 77 common autoantibodies. An examination of the AAgAtlas & PubMed revealed that 20 of our 77 common autoantibodies have been reported as disease-related biomarkers. These 20 common autoantibodies are STMN4, ODF2, RBPJ, AMY2A, EPCAM, ZNF688, CSF3, S1PR3, CDR2L, RASSF1, SPAG8, TRAP1, SART1, GATA2, PML, SOX2, PELI1, TAX1BP1, JUN, and PAK1.

TABLE 1
Weighted prevalence of common autoantibodies in healthy and diseased cohorts. Availability
of the autoantibodies in AAgAtlas/PubMed literature were reported as yes [Y] or no [N].

| | No. of | Total | Weighted | No. of | Total | Weighted | Found in |
|---|---|---|---|---|---|---|---|
| | reactivity | samples | prevalence | reactivity | samples | prevalence | AAgAtlas/ |
| Gene name | (Healthy) | (Healthy) | (Healthy) | (Diseased) | (Diseased) | (Diseased) | PubMed |
| STMN4 | 83 | 192 | 0.47 | 79 | 205 | 0.37 | Y |
| ODF2 | 85 | 178 | 0.42 | 82 | 182 | 0.38 | Y |
| RBPJ | 69 | 178 | 0.37 | 67 | 182 | 0.33 | Y |
| AMY2A | 71 | 167 | 0.34 | 66 | 169 | 0.33 | Y |
| EPCAM | 72 | 205 | 0.31 | 73 | 235 | 0.28 | Y |
| ZNF688 | 54 | 192 | 0.29 | 51 | 205 | 0.26 | Y |
| CSF3 | 62 | 259 | 0.25 | 54 | 293 | 0.19 | Y |
| RAD51AP1 | 30 | 178 | 0.23 | 33 | 182 | 0.24 | N |
| PSKH1 | 15 | 140 | 0.23 | 12 | 140 | 0.18 | N |
| LENG1 | 43 | 192 | 0.22 | 42 | 205 | 0.18 | N |
| S1PR3 | 62 | 269 | 0.21 | 57 | 314 | 0.17 | Y |
| LYSMD1 | 30 | 178 | 0.21 | 29 | 182 | 0.17 | N |
| FAM76A | 35 | 178 | 0.2 | 29 | 182 | 0.2 | N |
| CDR2L | 29 | 192 | 0.2 | 25 | 205 | 0.12 | Y |
| CCDC130 | 27 | 142 | 0.2 | 21 | 143 | 0.16 | N |
| SOX15 | 43 | 192 | 0.2 | 37 | 205 | 0.14 | N |
| PLA2G2A | 15 | 134 | 0.19 | 10 | 168 | 0.13 | N |
| TAF1D | 29 | 133 | 0.19 | 29 | 167 | 0.17 | N |
| CEP57L1 | 25 | 178 | 0.19 | 26 | 182 | 0.16 | N |
| RASSF1 | 26 | 140 | 0.18 | 20 | 140 | 0.14 | Y |
| PHF21A | 12 | 147 | 0.18 | 13 | 190 | 0.16 | N |
| POLDIP3 | 40 | 182 | 0.18 | 39 | 183 | 0.2 | N |
| ESS2 | 34 | 192 | 0.17 | 37 | 205 | 0.15 | N |
| C17orf80 | 32 | 182 | 0.17 | 22 | 183 | 0.12 | N |
| C9orf78 | 15 | 85 | 0.17 | 10 | 85 | 0.1 | N |
| MTUS2 | 16 | 178 | 0.16 | 15 | 182 | 0.15 | N |
| GDE1 | 9 | 140 | 0.16 | 6 | 140 | 0.12 | N |
| PMFBP1 | 25 | 192 | 0.16 | 22 | 205 | 0.17 | N |
| KAZN | 22 | 125 | 0.16 | 19 | 125 | 0.14 | N |
| SPAG8 | 14 | 182 | 0.16 | 8 | 183 | 0.1 | Y |
| CCDC144NL | 15 | 125 | 0.15 | 15 | 125 | 0.09 | N |
| SNRK | 21 | 140 | 0.15 | 26 | 140 | 0.18 | N |
| CCDC34 | 26 | 182 | 0.15 | 18 | 183 | 0.11 | N |
| MAP11 | 11 | 182 | 0.14 | 9 | 183 | 0.14 | N |
| TRAP1 | 8 | 139 | 0.14 | 5 | 143 | 0.09 | Y |
| SART1 | 21 | 182 | 0.14 | 16 | 183 | 0.09 | Y |
| CTTNBP2NL | 20 | 125 | 0.14 | 22 | 125 | 0.13 | N |
| KRT35 | 27 | 182 | 0.13 | 25 | 183 | 0.14 | N |
| WTAP | 8 | 178 | 0.13 | 10 | 182 | 0.1 | N |
| TCEAL4 | 21 | 182 | 0.13 | 27 | 183 | 0.16 | N |
| C19orf47 | 8 | 182 | 0.13 | 2 | 183 | 0.04 | N |
| GATA2 | 8 | 192 | 0.13 | 8 | 205 | 0.03 | Y |
| ZNF177 | 13 | 192 | 0.12 | 15 | 205 | 0.08 | N |
| PSMD2 | 18 | 140 | 0.12 | 22 | 140 | 0.16 | N |
| PML | 19 | 140 | 0.12 | 23 | 140 | 0.16 | Y |
| SOX2 | 27 | 192 | 0.12 | 30 | 205 | 0.12 | Y |
| MAK | 20 | 140 | 0.12 | 14 | 140 | 0.09 | N |
| FRG1 | 22 | 178 | 0.12 | 30 | 182 | 0.18 | N |
| ZSCAN16 | 20 | 192 | 0.12 | 23 | 205 | 0.11 | N |
| TRIM29 | 20 | 192 | 0.12 | 20 | 205 | 0.1 | N |
| PAK5 | 20 | 192 | 0.12 | 12 | 205 | 0.08 | N |
| PELI1 | 7 | 178 | 0.11 | 1 | 182 | 0.02 | Y |
| GTSE1 | 14 | 179 | 0.11 | 19 | 183 | 0.13 | N |
| MAPK13 | 31 | 259 | 0.11 | 29 | 293 | 0.11 | N |
| APEX2 | 27 | 259 | 0.11 | 13 | 293 | 0.04 | N |
| VPS72 | 11 | 192 | 0.11 | 13 | 205 | 0.05 | N |
| MYLK2 | 12 | 140 | 0.11 | 6 | 140 | 0.04 | N |
| TAX1BP1 | 6 | 140 | 0.11 | 7 | 140 | 0.12 | Y |
| LEF1 | 26 | 192 | 0.11 | 16 | 205 | 0.06 | N |
| AHCY | 6 | 192 | 0.1 | 2 | 205 | 0.04 | N |
| ADNP2 | 6 | 125 | 0.1 | 0 | 125 | 0 | N |
| RPS21 | 12 | 147 | 0.1 | 8 | 190 | 0.13 | N |
| TCEAL2 | 7 | 192 | 0.1 | 6 | 205 | 0.03 | N |
| RABGEF1 | 16 | 125 | 0.1 | 14 | 125 | 0.11 | N |
| TFAM | 10 | 125 | 0.1 | 10 | 125 | 0.1 | N |
| GPANK1 | 14 | 259 | 0.1 | 19 | 293 | 0.05 | N |
| CAPN3 | 12 | 178 | 0.1 | 10 | 182 | 0.1 | N |
| DTNA | 6 | 178 | 0.1 | 3 | 182 | 0.05 | N |
| ZCCHC10 | 6 | 178 | 0.1 | 9 | 182 | 0.07 | N |
| VENTX | 15 | 125 | 0.1 | 13 | 125 | 0.09 | N |
| NF2 | 14 | 179 | 0.1 | 11 | 183 | 0.06 | N |
| YJEFN3 | 15 | 182 | 0.1 | 13 | 183 | 0.07 | N |
| SECISBP2 | 17 | 182 | 0.1 | 15 | 183 | 0.09 | N |
| ZBTB22 | 13 | 140 | 0.1 | 7 | 140 | 0.06 | N |
| RNF138 | 11 | 125 | 0.1 | 11 | 125 | 0.06 | N |
| JUN | 7 | 272 | 0.1 | 5 | 315 | 0.05 | Y |
| PAK1 | 9 | 140 | 0.1 | 5 | 140 | 0.1 | Y |

As shown in the examples, a subset of common autoantibodies may be used as indicators of a false positive result in the cancer or autoimmunity screening. Thus, disclosed herein is a method of increasing the accuracy of a cancer or an autoimmune condition screening in a subject suspected of having cancer or an autoimmune condition by identifying at least one false positive indicator. The method comprises providing a biological sample from the subject; detecting the presence of one or more autoantibodies against at least one autoantigen listed in Table 1 in the biological sample, and identifying at least one false positive indicator upon detecting the presence of the one or more autoantibodies.

The biological sample from the subject can be any type of biological sample, for example, a tissue sample, a saliva sample, or a blood sample, including a fraction thereof such as a plasma sample or a serum sample. In certain embodiments, the biological sample is a serum sample or a plasma sample.

The step for detecting the presence of one or more autoantibodies against at least one autoantigen may be performed by methods well-established in the art, for example by immunoassay, where autoantibody capture molecules are immobilized on a solid support. For example, the presence of one or more autoantibodies against at least one autoantigen is detected using Enzyme Linked Immunosorbent Assay (ELISA), Protein microarrays, Western Blot or beads based immunoassay. In some implementation, the autoantigens are immobilized on a solid surface, in the immunoassay. In other implementation, peptide methods, such as peptide arrays and phage display, are used to detect the presence of one or more autoantibodies against at least one autoantigen.

In some implementations of the method of increasing the accuracy of a cancer or an autoimmune condition screening, the at least one autoantigen is selected from the group consisting of: AMY2A, SPAG8, GTSE1, MAK, RNF138, PMFBP1, CCDC34, ODF2, CSF3, CAPN3, MYLK2, TRIM29, SOX2, and STMN4. Where the biological sample is from the subject's pancreas, the at least one autoantigen is AMY2A. Where the biological sample is from the subject's lungs, the at least one autoantigen is CSF3. Where the biological sample is from the subject's salivary gland, nerve tibial, or brain, the at least one autoantigen is SOX2. Where the biological sample is from the subject's esophagus, skin, or vagina, the at least one autoantigen is TRIM29. Where the biological sample is from the subject's skeletal muscle, the at least one autoantigen is CAPN3 and/or MYLK2. Where the biological sample is from the subject's testis, the at least one autoantigen is selected from the group consisting of: SPAG8, GTSE1, MAK, RNF138, PMFBP1, CCDC34, and ODF2.

In other implementations of the method, the at least one autoantigen is selected from the group consisting of: STMN4, ODF4, RBPJ, AMY2A, EPCAM, ZNF688, CSF3, RAD51AP1, PSKH1, LENG1, S1PR3, LYSMD1, FAM76A, CDR2L, and CCDC130. In some embodiments, where the at least one autoantigen is EPCAM, the method further comprises detecting the presence of at least one other autoantibody against EDG3 or CSF3, wherein the presence of the at least one other autoantibody against EDG3 or CSF3 in the biological sample indicates the subject is false positive for having cancer or an autoimmune condition.

In some aspects, the method of increasing the accuracy of a cancer or an autoimmune condition screening comprises detecting at least two, at least three, at least four, or at least five autoantibodies against at least two, at least three, at least four, or at least five autoantigens selected from the group consisting of: STMN4, ODF4, RBPJ, AMY2A, EPCAM, ZNF688, CSF3, RAD51AP1, PSKH1, LENG1, S1PR3, LYSMD1, FAM76A, CDR2L, and CCDC130. Detecting the presence of the at least two, at least three, at least four, or at least five autoantibodies indicates that the subject is false positive for having cancer or an autoimmune condition. In certain embodiments, the method comprises detecting at least ten autoantibodies against at least ten selected from the group consisting of: STMN4, ODF4, RBPJ, AMY2A, EPCAM, ZNF688, CSF3, RAD51AP1, PSKH1, LENG1, S1PR3, LYSMD1, FAM76A, CDR2L, and CCDC130. Detecting the presence of the at least ten autoantibodies indicate that the subject is false positive for having cancer or an autoimmune condition. In a particular embodiment, wherein the method comprises detecting the presence of at least one autoantibody against STMN4, ODF4, RBPJ, AMY2A, EPCAM, ZNF688, CSF3, RAD51AP1, PSKH1, LENG1, S1PR3, LYSMD1, FAM76A, CDR2L, and CCDC130 in the biological sample, the presence of at least one autoantibody against STMN4, ODF4, RBPJ, AMY2A, EPCAM, ZNF688, CSF3, RAD51AP1, PSKH1, LENG1, S1PR3, LYSMD1, FAM76A, CDR2L, and CCDC130 in the biological sample indicate the subject is false positive for having cancer or an autoimmune condition.

In certain implementations, the method comprises detecting the presence of at least one autoantibody against at least one autoantigen selected from the group consisting of: STMN4, ODF2, RBPJ, AMY2A, EPCAM, and ZNF688 in the biological sample. Detecting the presence of the at least one autoantibody against STMN4, ODF2, RBPJ, AMY2A, EPCAM, or ZNF688 indicates the subject is false positive for having cancer or an autoimmune condition. In some aspects, the method comprises detecting the presence of at least two, at least three, at least four, or at least five autoantibodies against at least two, at least three, at least four, or at least five autoantigens selected from the group consisting of: STMN4, ODF2, RBPJ, AMY2A, EPCAM, and ZNF688 in the biological sample, wherein the presence of the at least two, at least three, at least four, or at least five autoantibodies indicates the subject is false positive for having cancer or an autoimmune condition. In particular embodiments, the method comprises detecting the presence of at least one autoantibody against STMN4, ODF2, RBPJ, AMY2A, EPCAM, and ZNF688 in the biological sample, wherein the presence of the at least one autoantibody against STMN4, ODF2, RBPJ, AMY2A, EPCAM, and ZNF688 in the biological sample indicates the subject is false positive for having cancer or an autoimmune condition.

Also described herein are a diagnostic kit and array for diagnosing and verifying an autoimmune condition or cancer in a subject. In some aspects, the kit and the array comprise at least one false positive indicator, wherein the false positive indicator comprising at least one autoantigen listed in Table 1. For example, the kit comprises at least one antigen or antibody targeting a cancer marker, an autoimmune marker, or both and a false positive indicator, the false positive indicator comprising at least one autoantigen listed in Table 1. In some embodiments, the kit further comprises at least one labeled secondary antibody or antigen for detecting the binding of the at least one antigen or antibody with the cancer marker and/or the autoimmune marker; and at least one labeled secondary antibody or antigen detecting binding of the false positive indicator to the at least one autoantigen. The diagnostic array comprises at least one antigen or antibody targeting a cancer marker, an autoimmune marker, or both and a false positive indicator, the false positive indicator being selected from at least one autoantigen listed in Table 1. In some aspects, wherein the cancer marker is P53. In other aspects, the autoimmune marker is antinuclear antibodies.

### EXAMPLES

The present invention is further illustrated by the following examples that should not be construed as limiting. The contents of all references, patents, and published patent applications cited throughout this application, as well as the Figures, are incorporated herein by reference in their entirety for all purposes.

### I. Identity and Prevalence of Common Autoantibodies

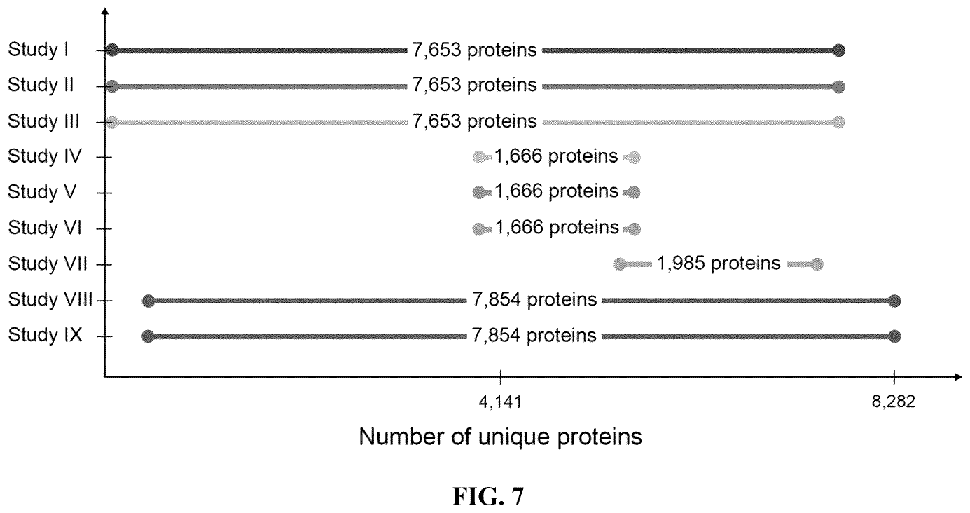

Autoantibody profiles for 272 healthy subjects from 9 case-control studies were compiled (Table 2). There were more females than males, 195 vs. 67, because several studies focused on female-specific diseases such as breast and ovarian cancers. These studies were diverse in terms of subject ages, ranging from infancy to adulthood, with most above 50 years old. Antibodies against 8,282 unique human proteins were studied; although, the number of proteins studied for each subject varied by study (FIG. 7). The study used subjects from different studies performed at different times, some with smaller protein subsets, and an overall moderate number of samples. While these factors do not limit the validity of the common autoantibodies found here, they limit the statistical power for finding less prevalent ones. There were more samples from female than male participants. Direct comparison revealed no difference in the identity of common antibodies in male subjects.

To minimize the effect of study heterogeneity, sample size based weighted prevalence was calculated as the sum of individual prevalence of antibody in each study multiplied by the sample size of the study. For the healthy subjects, 77 autoantibodies occurred frequently and had a weighted prevalence between 10%-47% (Table 1). These were termed as common autoantibodies. Antibodies against STMN4, ODF2, RBPJ, AMY2A, EPCAM, and ZNF688 showed the highest prevalence (Table 1).

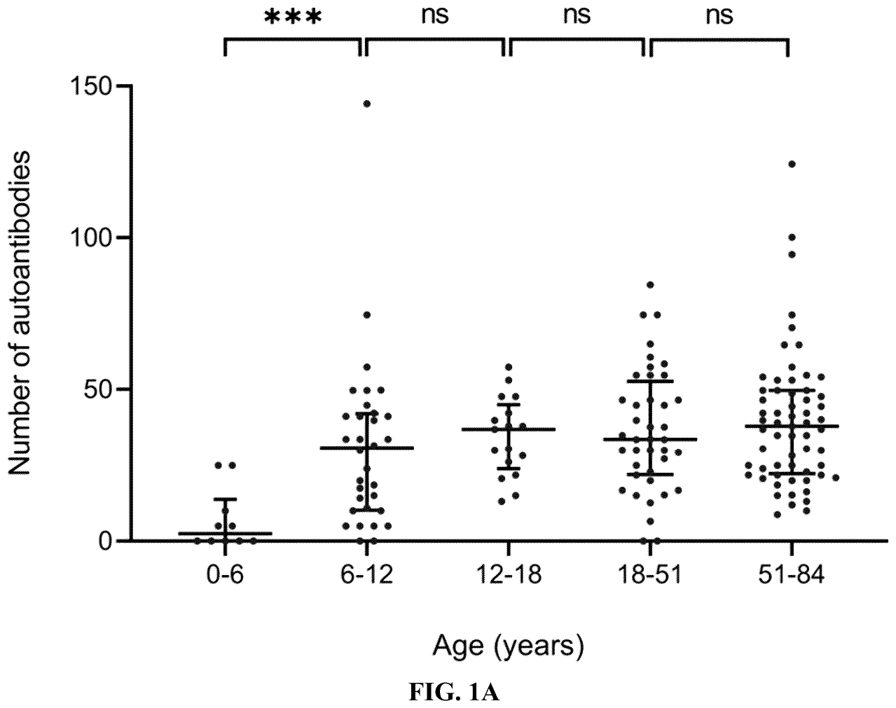

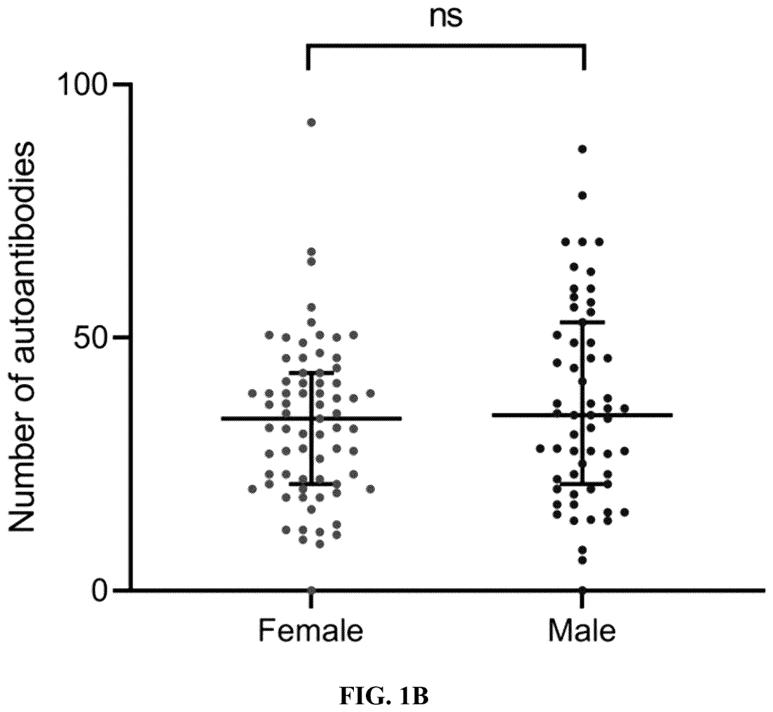

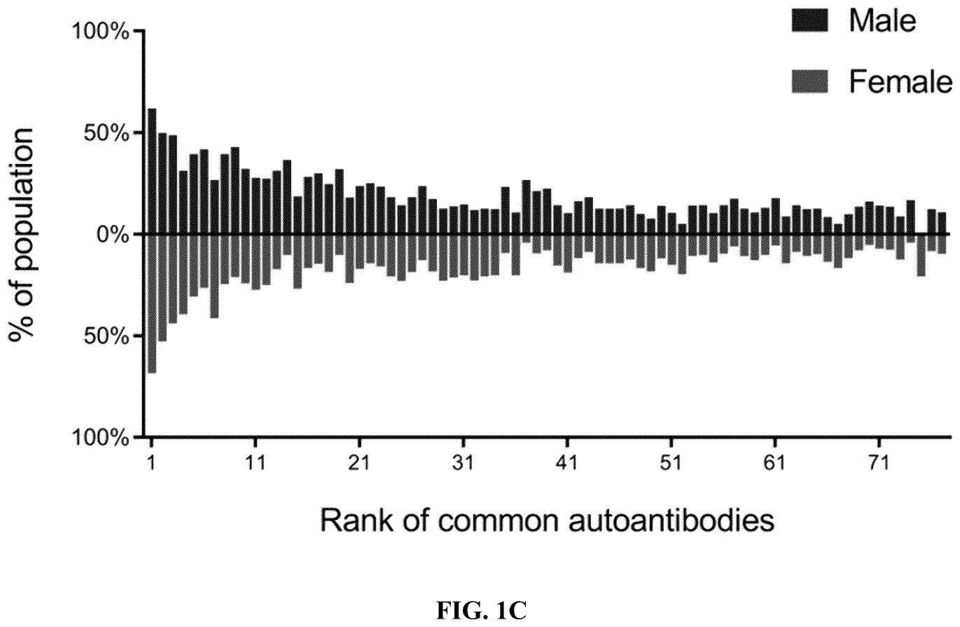

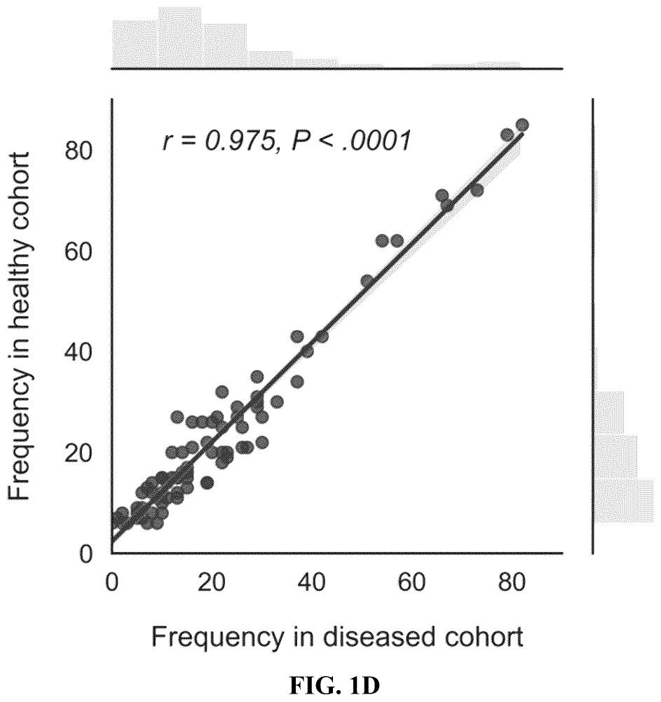

160 healthy subjects from five studies that included age information (Studies I, II, IV, VI, VII, Table 2) were divided into five age groups based on human development stages to examine the time course of autoantibody development. The infant and early childhood age group (0-6 years) had the least number of autoantibodies. The number increased in the middle and late childhood age group (6-12 years) and then plateaued (FIG. 1A, P<0.001). To investigate whether the number or identity of autoantibodies showed a gender bias, we compiled four studies that included both male and female subjects with matched age (Studies I, II, IV, VII) and compared the counts and identities of the antibodies. The median numbers of autoantibodies for male and female subjects were similar (FIG. 1B, P=0.17). The weighted prevalence of 77 common autoantibodies also had comparable distribution between male and female subjects (FIG. 1C, P=0.06).

If these common autoantibodies observed in the healthy subjects were elicited through common non-pathogenic mechanisms, then they should also occur at similar frequencies in their matched disease cohorts. Indeed, the 77 common autoantibodies occurred at similar frequencies in diseased cohorts to those in healthy cohorts (FIG. 1D, Pearson correlation coefficient r=0.975).

TABLE 2
Study summary with demographic information. Each study was performed independently
with age and gender matched case-control samples. Some studies focused
on female-specific diseases without samples from male subjects.

| | Healthy | Diseased | Healthy | Healthy | Age | No. of proteins | |
|---|---|---|---|---|---|---|---|
| Study | subjects | subjects | Male | Female | (median) | studied | Reference |
| I | 40 | 40 | 14 | 26 | 13 | 7,653 | (Bian et al., |
| | | | | | | | 2016) |
| II | 40 | 40 | 19 | 21 | 71.5 | 7,653 | (J. Wang et al., |
| | | | | | | | 2016) |
| III | 45 | 45 | 0 | 45 | — | 7,653 | (Wang et al., |
| | | | | | | | 2015a) |
| IV | 40 | 40 | 21 | 19 | 51 | 1,666 | |
| V | 10 | 20 | — | — | — | 1,666 | (Wang et al., |
| | | | | | | | 2017) |
| VI | 30 | 50 | 8 | 22 | 7.11 | 1,666 | |
| VII | 10 | 21 | 5 | 5 | 51.5 | 1,985 | |
| VIII | 30 | 30 | 0 | 30 | — | 7,854 | (Katchman et |
| | | | | | | | al., 2017) |
| IX | 27 | 29 | 0 | 27 | 50 | 7,854 | |
| Total | 272 | 315 | | | | 8282 (unique) | |

“—” represents “data not available”.

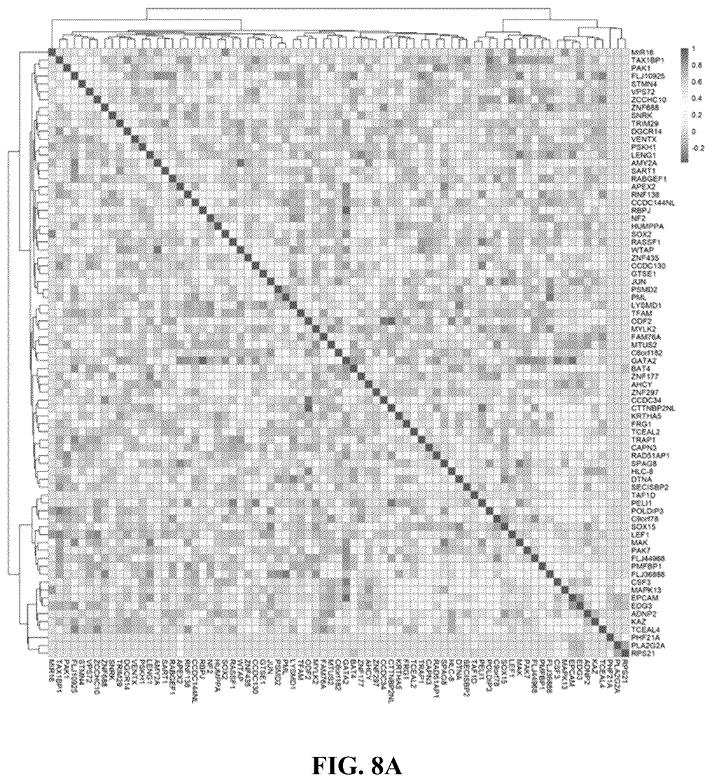

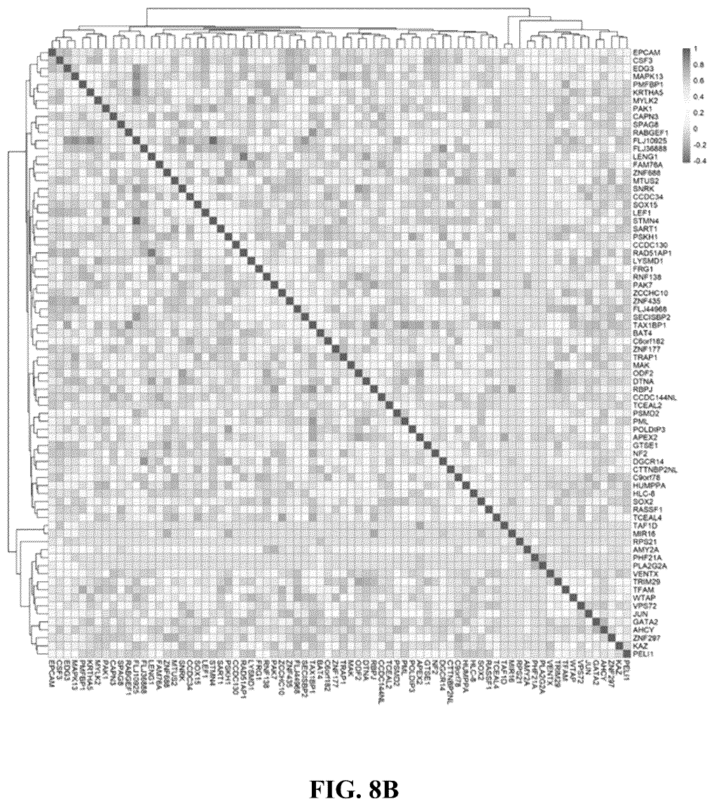

To answer whether any of these common autoantibodies were related to each other (in other words, was there any concordance among them or were their occurrences independent), the common autoantigens were analyzed pairwise to determine if any occur together in healthy individuals at frequencies greater than chance alone (FIG. 8A). The majority of them were independent of each other except several pairs: EDG3 and EPCAM (Phi correlation coefficient: 0.83), PML and PSMD2 (Phi correlation coefficient: 0.73), and EPCAM and CSF3 (Phi correlation coefficient: 0.67). Moreover, their concordance was also elevated in diseased individuals (FIG. 8B). Table 3 shows the pairs that have correlation coefficient higher than 0.6 in healthy cohort and that have correlation in at least two studies were shown with their corresponding value in diseased cohort.

TABLE 3
Pairs that have correlation coefficient higher than 0.6 in both the
cohorts are shown in bold. P value was calculated using a normal
approximation from the “metacor” function in the R meta package.

| | | Phi correlation | Phi correlation | Number of studies | |
|---|---|---|---|---|---|
| Alias1 | Alias2 | coefficient (healthy) | coefficient (diseased) | having correlation | P value |
| EDG3 | EPCAM | 0.83 | 0.67 | 6 | <0.001 |
| RPS21 | PLA2G2A | 0.76 | 0.53 | 2 | <0.001 |
| PML | PSMD2 | 0.73 | 0.63 | 3 | <0.001 |
| PLA2G2A | EPCAM | 0.69 | 0.44 | 2 | <0.001 |
| PLA2G2A | EDG3 | 0.68 | 0.19 | 2 | <0.001 |
| CSF3 | EPCAM | 0.67 | 0.65 | 6 | <0.001 |
| RPS21 | EDG3 | 0.66 | 0.58 | 3 | <0.001 |
| PHF21A | EPCAM | 0.63 | 0.31 | 2 | <0.001 |
| RPS21 | EPCAM | 0.63 | 0.52 | 3 | <0.001 |

### II. Sequence Similarity with Viral Proteins

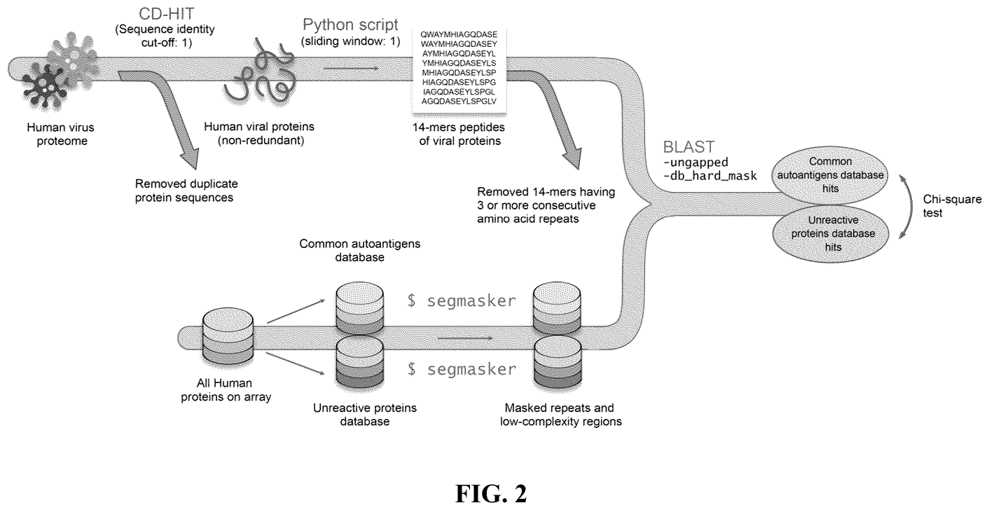

To understand the extent that common autoantibodies observed in our study resulted from cross-reactivity of antibodies induced by viral infection, the sequence similarities between viral proteins and common autoantigens was examined. As these autoantibodies developed early in age and did not change after adolescence, respiratory and common viruses found in children of the US were included in the analysis (Table 4). In order to avoid redundancy and false positives, duplicate proteins and consecutive amino acid repeats were removed from viral proteomes (FIG. 2). Similarly, human proteins were masked to avoid repeats and low-complexity regions (homopolymeric runs, short-period repeats and over representation of one or few residues) as potential hits. Using 7 ungapped amino acids match as the threshold, 28 instances of 7 ungapped amino acid matches and 1 instance of 8 ungapped amino acid match with viral proteins that were present in 21 common autoantigens (Table 5) were identified. Some of the matches were from the peptides of high-complexity regions like SYFGLRT (SEQ ID NO. 1), LRQEINA (SEQ ID NO. 2), WPEGYQL (SEQ ID NO. 3), and ARCETQN (SEQ ID NO. 4). To assess if these matches were statistically significantly higher than random chance, the total sequence matches above the threshold for the unreactive proteins (i.e., proteins without any autoantibody response) against the same set of viral proteins was calculated. To control for increased chance of a match due to protein length, the results were normalized and expressed as frequency at amino acid level. There were 201 amino acids in matched peptides higher than the threshold among 34,070 amino acids of the common autoantigens while 5,801 amino acids matched higher than the threshold among 2,026,890 amino acids of the unreactive proteins (Chi-square test, P<0.00001).

TABLE 4
Viral strains and number of viral proteins used for sequence similarity analysis.
The analysis relates to FIG. 2 and Table 5. Following strains of respiratory
and common viruses found in children of the US were used for the analysis.
The reference proteomes from UniProt were included for each virus.

| | | Number of |
|---|---|---|
| Organism | Strains | proteins |
| Influenza A virus | (A/Alaska/105/2015(H3N2)), | 218 |
| | (A/Boston/151/2009(H1N1)), | |
| | (A/Boston/DOA29/2011(H3N2)), | |
| | (A/Boston/YGA_01042/2012(H3N2)), | |
| | (A/California/47/2016(H3N2)), | |
| | (A/California/VRDL67/2009(H1N1)), | |
| | (A/California/VRDL364/2009(mixed)), | |
| | (A/Hawaii/67/2014(H1N1)), | |
| | (A/Hawaii/74/2015(H3N2)), | |
| | (A/Houston/JMM_42/2012(H3N2)), | |
| | (A/Kentucky/16/2015(H1N1)), | |
| | (A/Louisiana/13/2014(H3N2)), | |
| | (A/New York/169/2000(H3N2)), | |
| | (A/New York/441/2001(H1N1)), | |
| | (A/New York/1144/2008(H3N2)), | |
| | (A/New York/3052/2009(mixed)), | |
| | (A/New York/WC- LVD-14- | |
| | 057/2014(H1N1)), | |
| | (A/Oregon/29/2015(H1N1)), | |
| | (A/South Carolina/09/2009(H1N1)), | |
| | (A/Tennessee/F2019A/2011(H3N2)), | |
| | (A/Utah/06/2016(H1N1)), | |
| | (A/Virginia/43/2016(H3N2)), | |
| | (A/Puerto Rico/8/1934 H1N1), | |
| | (A/South Carolina/1/1918 H1N1)), | |
| | (A/WS/1933 H1N1)), | |
| | (swl A/California/04/2009 H1N1) | |
| Influenza B virus | (B/Florida/66/2015), | 69 |
| | (B/Florida/78/2015), | |
| | (B/Texas/14/1991), | |
| | (B/Utah/15/2015), | |
| | (B/Florida/78/2015), | |
| | (B/Utah/31/2016), (B/Lee/1940) | |
| Influenza C virus | (C/Ann Arbor/1/1950) | 8 |
| Herpes simplex virus 1 | (strain 17) (HHV-1) | 73 |
| Varicella-zoster virus | (strain Dumas) (HHV-3), | 140 |
| | (strain Oka vaccine) (HHV-3) | |
| Epstein-Barr virus | (strain AG876) (HHV-4), | 187 |
| | (strain B95-8) (HHV-4), | |
| | (strain GD1) (HHV-4) | |
| Human cytomegalovirus | (strain AD169) (HHV-5), | 525 |
| | (strain Merlin) (HHV-5) | |
| Human B lymphotropic virus | (strain Uganda-1102) (HHV-6 variant A), | 205 |
| | (strain Z29) (HHV-6 variant B) | |
| Human T lymphotropic virus | (strain JI) (HHV-7) | 102 |
| Human rhinovirus A | (strain 41467-Gallo) (HRV-89) | 1 |
| Human rotavirus A, B, C, G9P | | 45 |
| Human adenovirus 21, 21a, 26, 52, 55, | (HAdV-18), (HAdV-7), (HAdV-1), | 599 |
| 56, A, B, C, D, D, E, F | (HAdV-2), (HAdV-5), (HAdV-17), | |
| | (HAdV-4), (HAdV-41) | |
| Human parainfluenza virus 1, 2, 4a | (strain Washington/1964) | 24 |
| Human respirovirus 1, 3 | | 10 |
| Respiratory syncytial virus A, B | (strain A2), | 44 |
| | (strain S-2) (HRSV-S2), | |
| | (strain B1) | |
| Norwalk virus | (strain GI/Human/United | 3 |
| | States/Norwalk/1968) | |
| Human Enterovirus | (strain USA/BrCr/1970) (EV71), | 11 |
| | (EV68) (EV-68), | |
| | Human parechovirus 2 (strain Williamson) | |
| | (HPeV-2), | |
| | Coxsackievirus A16, | |
| | Coxsackievirus B2 (strain Ohio-1), | |
| | Coxsackievirus B3 (strain Nancy), | |
| | Coxsackievirus B4 (strain | |
| | JVB/Benschoten/New York/51) | |
| Human metapneumovirus | (strain CAN97-83) (HMPV) | 9 |
| Total | | 2,273 |

TABLE 5
Sequence similarity of common autoantigens and viral proteins. Common autoantigens
with 7 or more ungapped amino acids that match with viral proteins are reported along with virus
name and the corresponding sequences.

| S. No. | Autoantigen | Viral UniProt ID | Sequence similarity | Organism |
|---|---|---|---|---|
| 1 | ADNP2 | P16812 | LPVPPGG | Human herpesvirus 5 |
| | | | LPVPPGG | |
| | | | (SEQ ID NO. 5) | |
| | | H9C1C1 | SYFGLRT | Human rotavirus C |
| | | | SYFGLRT | |
| | | | (SEQ ID NO. 1) | |
| 2 | AHCY | F8WQQ3 | GKLNVKL | Human adenovirus 41 |
| | | | GKLNVKL | |
| | | | (SEQ ID NO. 6) | |
| 3 | AMY2A | P16766 | SAGTSST | Human herpesvirus 5 |
| | | | SAGTSST | |
| | | | (SEQ ID NO. 7) | |
| 4 | APEX2 | M1JRT8 | NRSGYSG | Influenza A virus |
| | | | NRSGYSG | |
| | | | (SEQ ID NO. 8) | |
| | | P09289 | ALLAAGS | Human herpesvirus 3 |
| | | | ALLAAGS | |
| | | | (SEQ ID NO. 9) | |
| 5 | C9orf78 | P16764 | EDCLYEL | Human herpesvirus 5 |
| | | | EDCLYEL | |
| | | | (SEQ ID NO. 10) | |
| 6 | CTTNBP2NL | P52529 | EQLRAKL | Human herpesvirus 6A |
| | | | EQLRAKL | |
| | | | (SEQ ID NO. 11) | |
| | | C4AL53 | AKLNREE | Influenza A virus |
| | | | AKLNREE | |
| | | | (SEQ ID NO. 12) | |
| | | Q6SW92 | SSNTVVA | Human herpesvirus 5 |
| | | | SSNTVVA | |
| | | | (SEQ ID NO. 13) | |
| 7 | FLJ36888 | P52355 | TIKRTLV | Human herpesvirus 7 |
| | | | TIKRTLV | |
| | | | (SEQ ID NO. 14) | |
| 8 | KAZ | 009800 | ARCETQN | Human herpesvirus 1 |
| | | | ARCETQN | |
| | | | (SEQ ID NO. 15) | |
| 9 | MAK | P16793 | GTSEVDE | Human herpesvirus 5 |
| | | | GTSEVDE | |
| | | | (SEQ ID NO. 16) | |
| | | Q01350 | WPEGYQL | Human herpesvirus 6A |
| | | | WPEGYQL | |
| | | | (SEQ ID NO. 17) | |
| | | Q69513 | KSDSELS | Human herpesvirus 7 |
| | | | KSDSELS | |
| | | | (SEQ ID NO. 18) | |
| 10 | MAPK13 | Q8QT31 | VIGLLDV | Human parainfluenza |
| | | | VIGLLDV | virus 1 |
| | | | (SEQ ID NO. 19) | |
| 11 | MTUS2 | P09284 | IDQNTVV | Human herpesvirus 3 |
| | | | IDQNTVV | |
| | | | (SEQ ID NO. 20) | |
| | | A0A0D5Z8N5 | SPIKLSP | Rotavirus B |
| | | | SPIKLSP | |
| | | | (SEQ ID NO. 21) | |
| 12 | MYLK2 | Q6SWDO | TAEEGKNI (SEQ ID | Human herpesvirus 5 |
| | | | NO. 22) | |
| | | | KAEEGKNI (SEQ | |
| | | | ID NO. 23) | |
| 13 | PAK1 | P24433 | SVIEPLP | Human herpesvirus 6A |
| | | | SVIEPLP | |
| | | | (SEQ ID NO. 24) | |
| 14 | PAK7 | P16739 | NATAQELL (SEQ | Human herpesvirus 5 |
| | | | ID NO. 25) | |
| | | | RATAQELL (SEQ | |
| | | | ID NO. 26) | |
| 15 | PELI1 | Q9QJ30 | LRQEINA | Human herpesvirus 6B |
| | | | LRQEINA | |
| | | | (SEQ ID NO. 2) | |
| 16 | PML | A0MK42 | TLGAVVP | Human adenovirus 52 |
| | | | TLGAVVP | |
| | | | (SEQ ID NO. 27) | |
| 17 | RABGEF1 | I1V183 | SPRKQEAE | Human adenovirus 7 |
| | | | SPRKQEAE | |
| | | | (SEQ ID NO. 28) | |
| 18 | SECISBP2 | D3JIS2 | ELTVAAR | Human adenovirus 18 |
| | | | ELTVAAR | |
| | | | (SEQ ID NO. 29) | |
| 19 | TAF1D | P09252 | DATHLED | Human herpesvirus 3 |
| | | | DATHLED | |
| | | | (SEQ ID NO. 30) | |
| 20 | TRAP1 | POC723 | ALIRKLRQ (SEQ ID | Epstein-Barr virus |
| | | | NO. 31) | |
| | | | ALIRKLRD (SEQ ID | |
| | | | NO. 32) | |
| | | P10200 | AQLGPRR | Human herpesvirus 1 |
| | | | AQLGPRR | |
| | | | (SEQ ID NO. 33) | |
| 21 | ZNF688 | Q1HVD1 | GAQPPAP | Epstein-Barr virus |
| | | | GAQPPAP | |
| | | | (SEQ ID NO. 34) | |

### III. Biochemical and Structural Properties

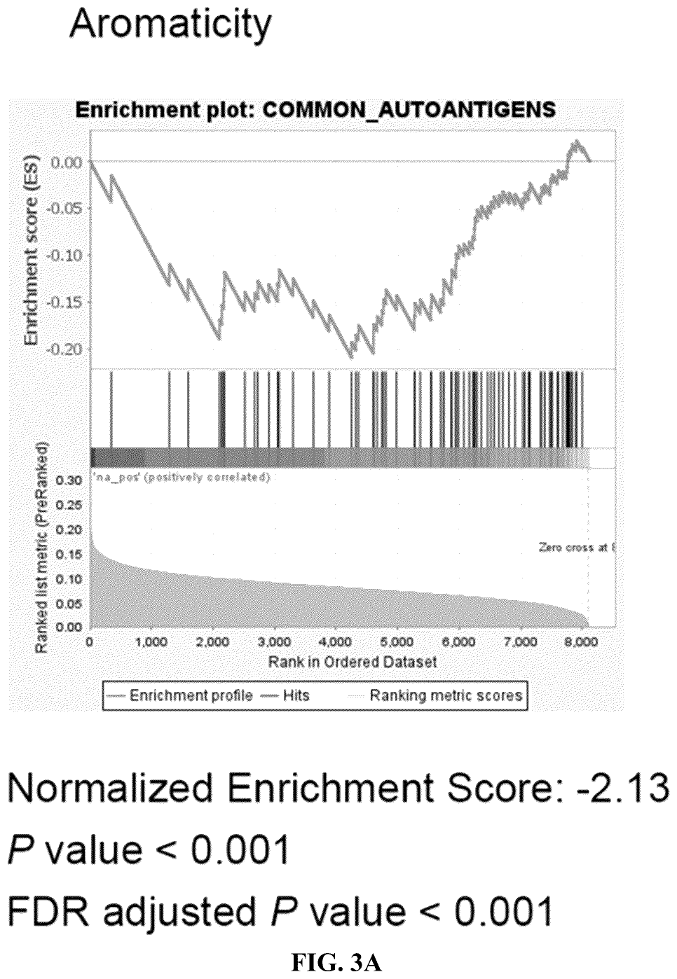

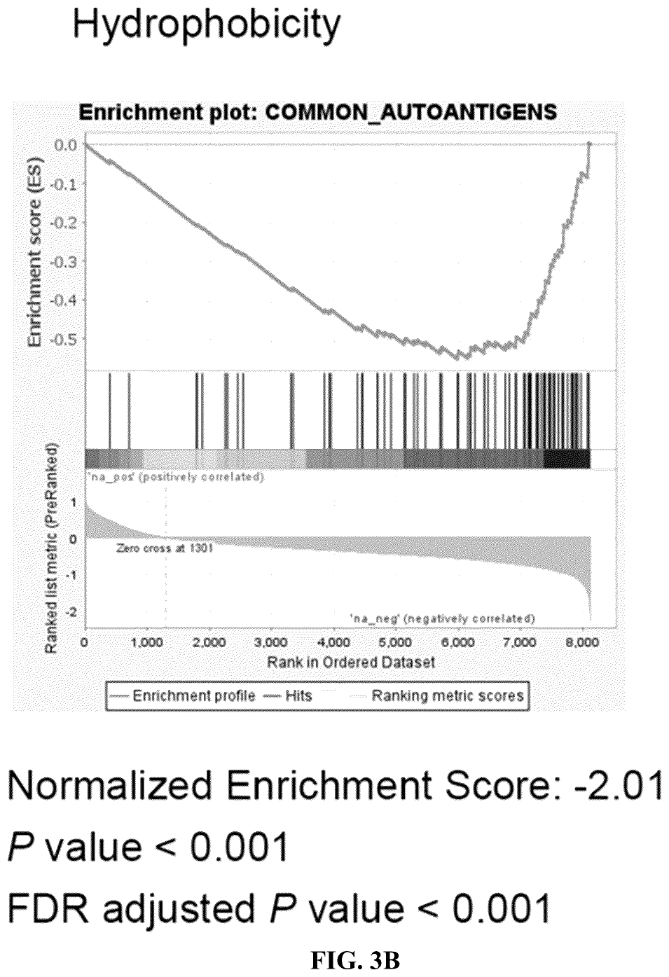

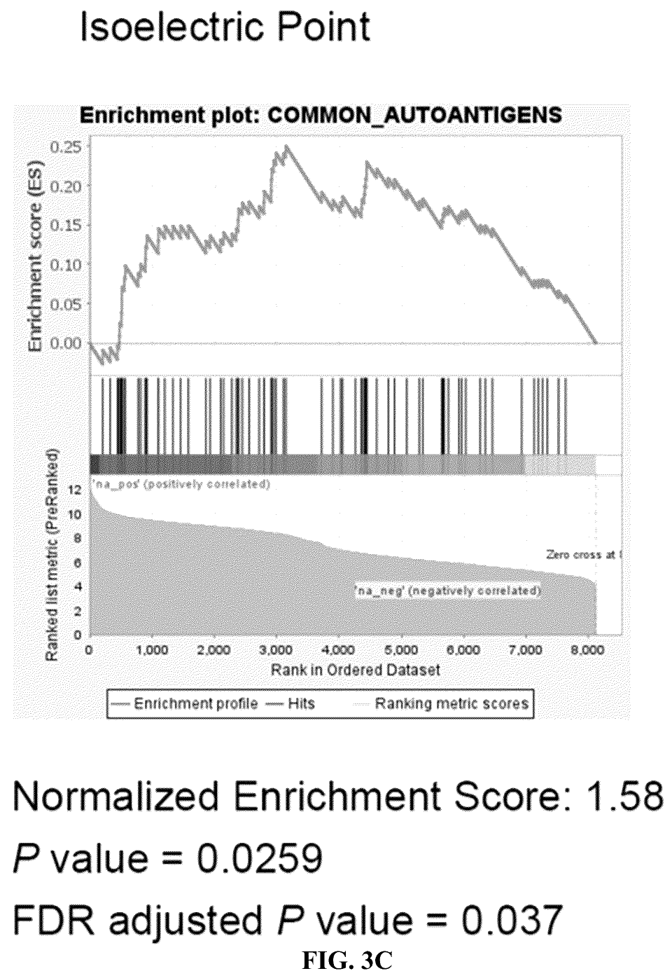

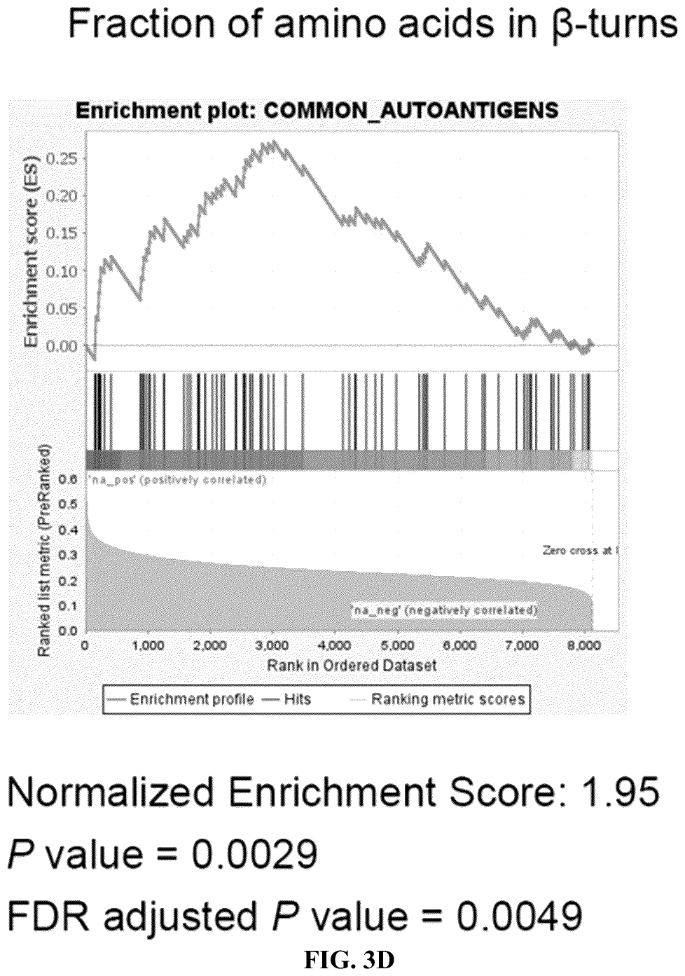

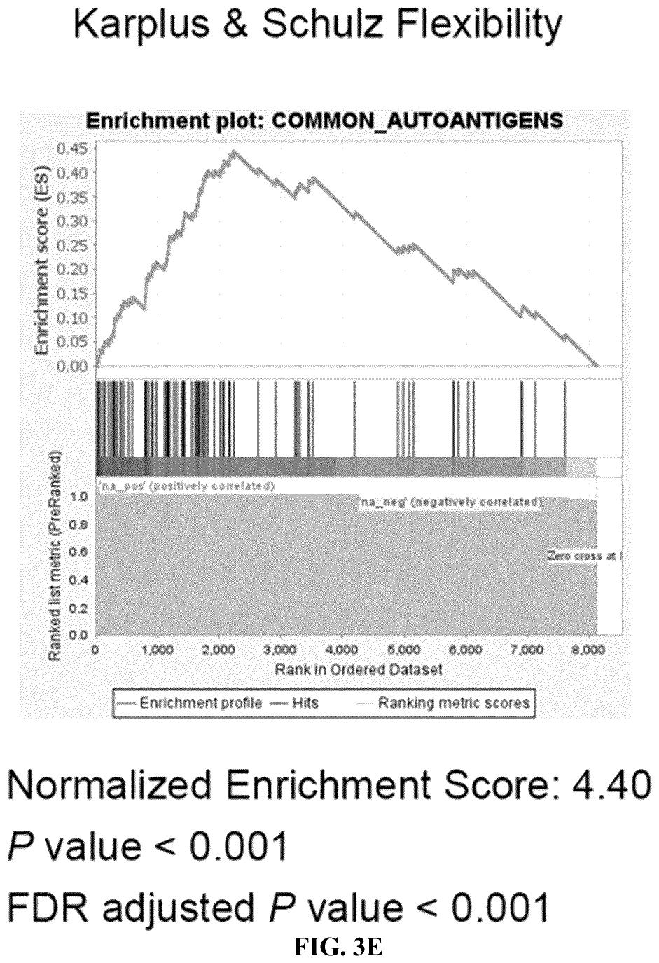

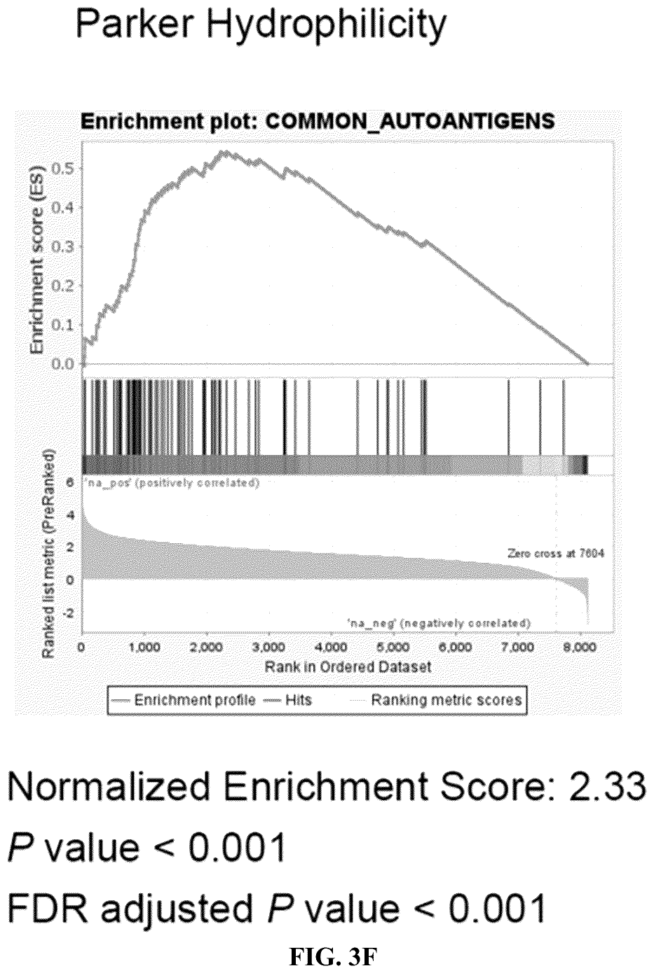

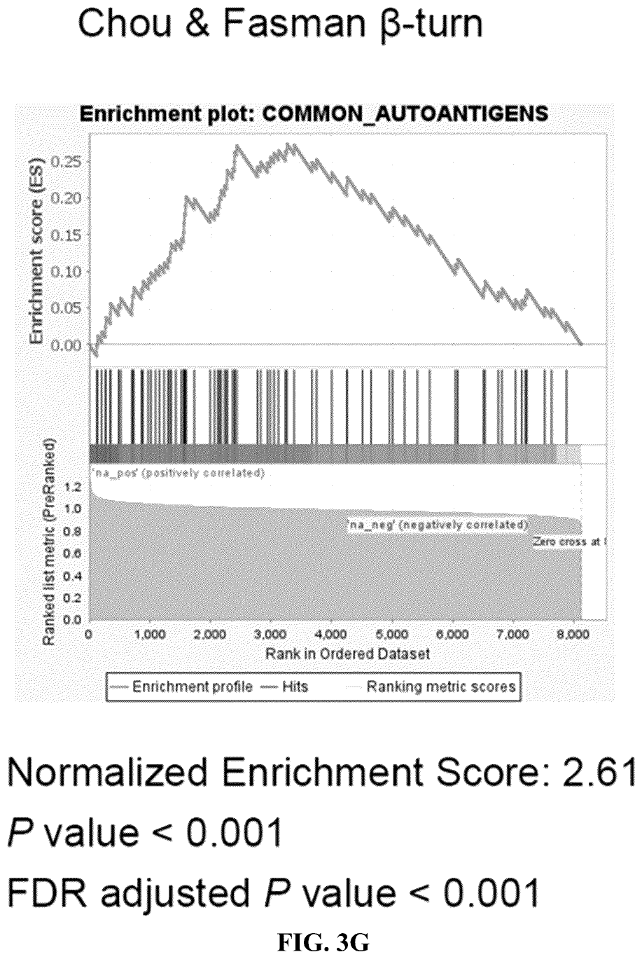

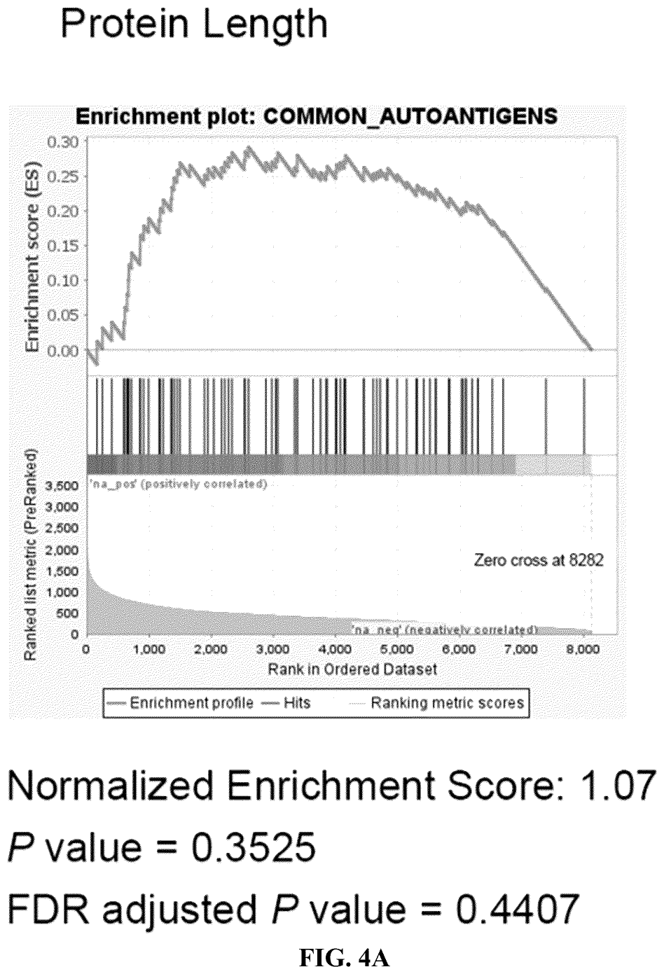

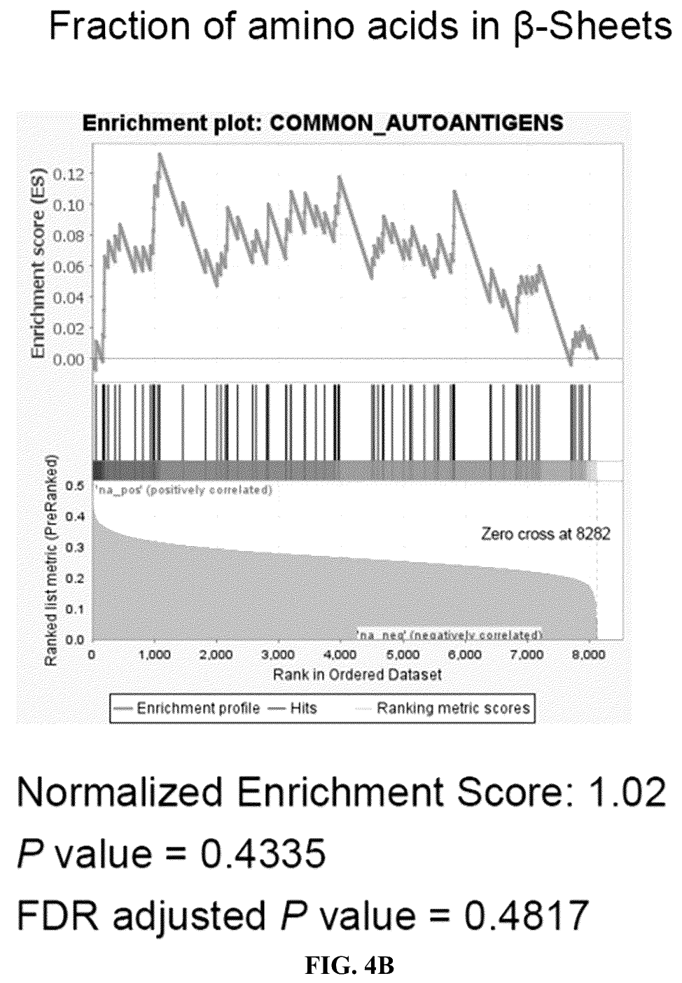

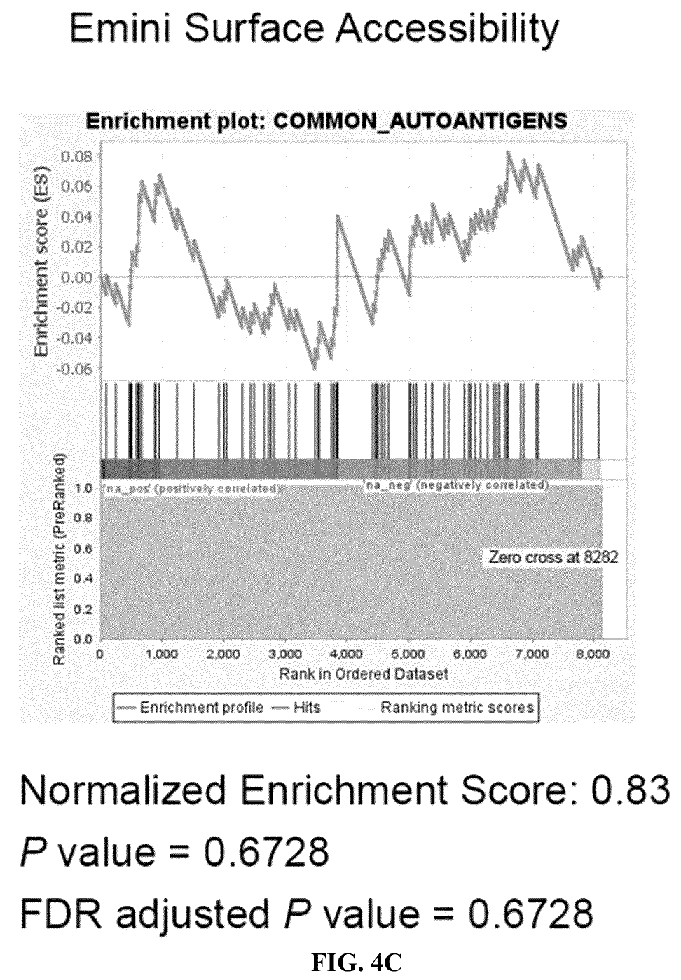

To determine whether any intrinsic biochemical and structural properties of the target antigens were responsible for common autoantibodies production, various properties were examined by comparing our list of common autoantigens to all 8,282 proteins using Gene Set Enrichment Analysis (GSEA). The 77 common autoantigens were significantly enriched with proteins having low aromaticity (NES or normalized enrichment score: −2.13, P<0.001), low hydrophobicity (NES: −2.01, P<0.001), high isoelectric point (NES: 1.58, P=0.018), high fraction of amino acids in β-turns (NES: 1.95, P=0.04), high Karplus & Schulz flexibility (NES: 4.40, P<0.001), high Parker hydrophilicity (NES: 2.33, P<0.001), and high Chou & Fasman β-turn score (NES: 2.61, P<0.001) (FIGS. 3A-3G). However, other biochemical properties such as protein length, the fraction of amino acids in β-sheets, and Emini surface accessibility showed no significant enrichment (FIGS. 4A-4C).

### IV. Subcellular Localization and Tissue Expression

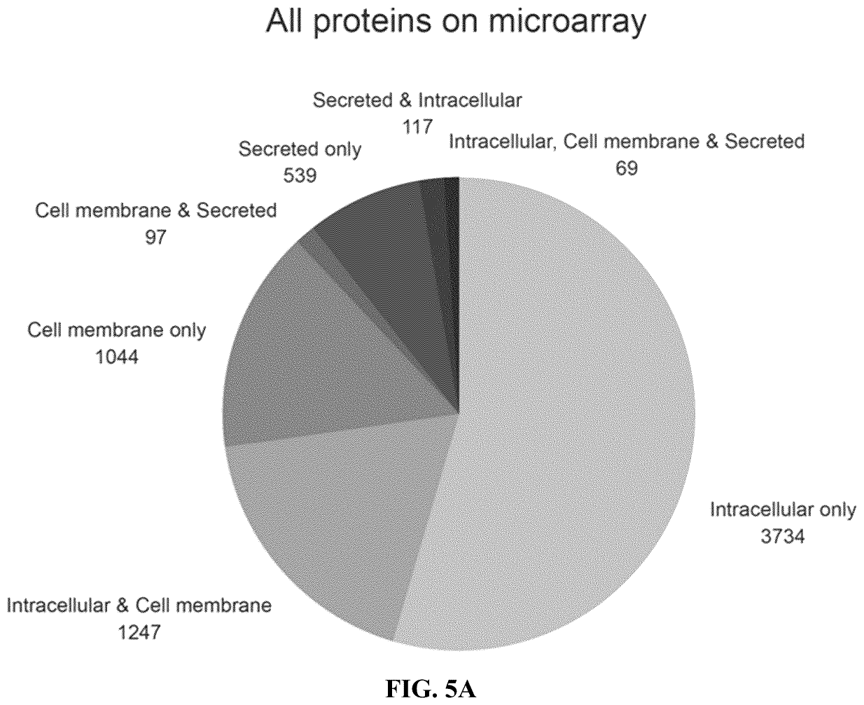

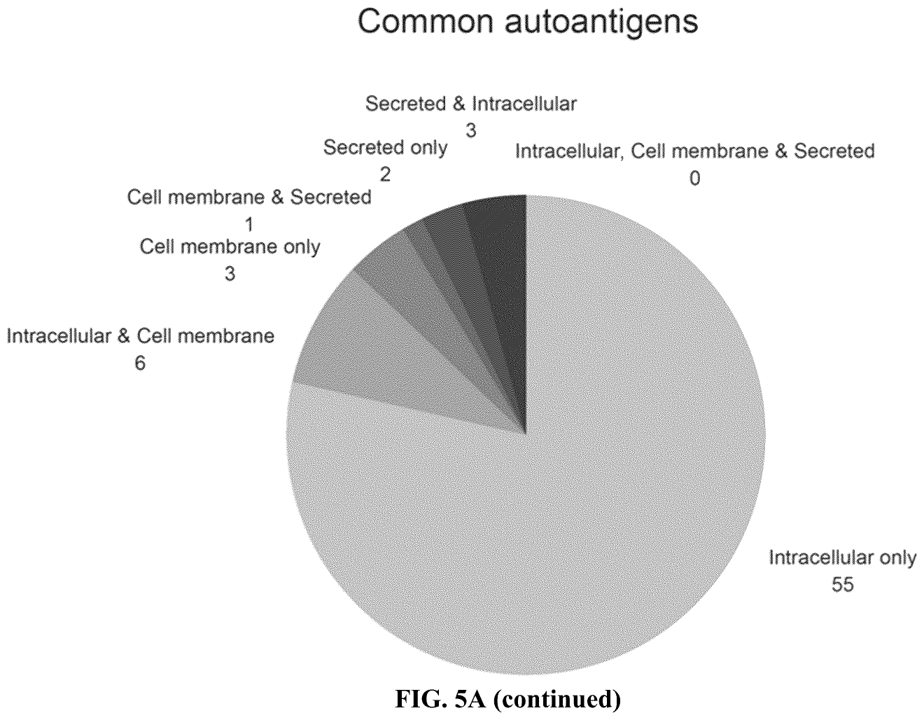

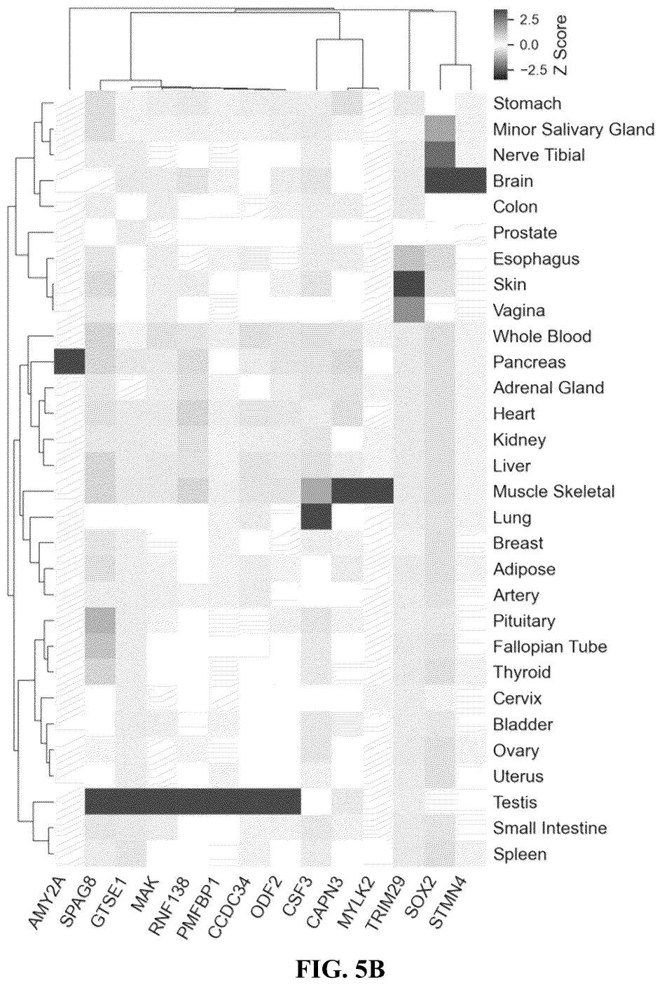

The discovery of common autoantibodies in healthy individuals raised the question about why these antibodies do not lead to autoantibody-mediated pathology. A primary requirement for such pathology is the formation of immune complexes. The subcellular localization of the common autoantigens to see if they were antibody accessible. They were divided into three broad categories: “intracellular”, “cell membrane”, and “secreted” (Table 6). The localization of an autoantigen can belong to one or more of these 3 categories. 55 among 70 common autoantigens were located exclusively at intracellular sites. The percentage of common autoantigens with “intracellular only” subcellular localization was significantly higher than that for all the proteins studied on the microarrays (78% vs. 54%, P<0.001) (FIG. 5A).

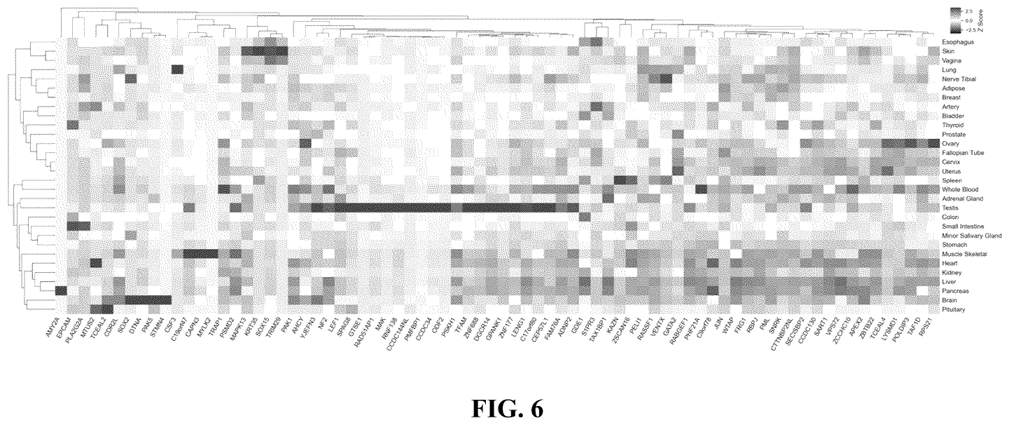

Tissue-specific gene expression can impact autoantigen exposure to circulating autoantibodies and the potential to trigger autoimmune disease. The data from GTEx, which is a public resource portal for tissue-specific gene expression in multiple human tissues, was used. In the GTEx dataset, transcripts encoding for 14 common autoantigens were organ/tissue-specific (defined as having log2 ((organ expression)/(mean expression in all other organs)>3) (FIG. 5B). Among them, PMFBP1, ODF2, RNF138, and CCDC34 were predominately expressed in testis while STMN4 and SOX2 were predominantly expressed in the brain. For instance, PMFBP1 has 29.47 TPM (transcripts per million) in testis while the mean in other organs is 0.48 TPM. Similarly, STMN4 has 77.23 TPM in the brain while the mean in other organs is 0.32 TPM. Other common autoantigens did not show tissue specificity (FIG. 6).

### V. STAR★Methods

TABLE 7
Key resources table

| | | |
|---|---|---|
| REAGENT or | | |
| RESOURCE | SOURCE | IDENTIFIER |

Deposited Data

| | | |
|---|---|---|
| Autoantibody | Mendeley Data | 10.17632/g57436wy6j.1 |
| reactivity raw | | |
| binary data | | |

Software and Algorithms

| | | |
|---|---|---|
| Array-Pro | Media Cybernetics | N/A |
| Analyzer 6.3 | | |
| Prism 9 | GraphPad | https://graphpad.com/ |
| R 3.5 | R Foundation | https://r-project.org/ |
| RStudio | RStudio PBC | https://rstudio.com/ |
| Python 3.7.6 | Python Software Foundation | https://python.org/ |
| Spyder 4.1.4 | Spyder project contributors | https://spyder-ide.org/ |
| Anaconda | Anaconda Inc. | https://anaconda.com/ |
| 1.9.12 | | |
| CD-HIT | Weizhong Li's group | http://cd-hit.org/ |
| MobaXterm | Mobatek | https://mobaxterm.mobatek.net/ |
| 20.1 | | |
| BLAST | National Center for | https://blast.ncbi.nlm.nih.gov/ |
| 2.10.1 | Biotechnology Information | |
| IEDB | National Institute of Allergy and | http://iedb.org/ |
| | Infectious Disease | |
| GSEA 4.2 | UC San Diego and Broad | https://gsea-msigdb.org/ |
| | Institute | |
| DAVID 6.8 | Laboratory of Human | https://david.ncifcrf.gov/ |
| | Retrovirology and | |
| | Immunoinformatics | |
| GTEx 8 | GTEx Consortium | https://gtexportal.org/ |
| UniProt | UniProt Consortium | https://uniprot.org/ |
| DNASU | Arizona State University | https://dnasu.org/ |
| Photoshop | Adobe Inc. | https://www.adobe.com/products/photoshop.html |

### VI. Method Details

a. Datasets

The healthy subjects included in this study were originally included in 9 different case-control studies (Table 2). These studies were all conducted in our lab; 5 of them were published (Table 2). The serum samples were collected from various parts of the USA and the UK. The goal of the original studies was to discover biomarkers of various cancers and autoimmune diseases by comparing the prevalence of antibodies present in diseased and healthy subjects. The presence of antibody was determined using protein microarrays that displayed thousands of human proteins as potential targets. Serum samples were probed on protein microarrays followed by a secondary antibody with a fluorophore tag specific for human IgG. Microarrays were scanned by a laser scanner. The microarray images from the 9 studies were qualitatively examined to identify protein targets that serum antibodies bound using Array-Pro Analyzer 6.3 (Media Cybernetics). All proteins were not probed by all samples included in our analysis (FIG. 7). Several studies focused on female-associated disease and thus only employed samples from females. A table of 8,282 rows of unique proteins and 587 columns of subjects in the case and control groups with binary response data of protein microarrays was created for data and statistical analysis (DOI: 10.17632/g57436wy6j.1).

b. Age and Gender Comparison

To understand the effect of age on autoantibody counts in healthy individuals, studies having both male and female subjects with age information were used (Studies I, II, IV, VI, VII, Table 2). A total of 160 subjects were divided into five age groups based on human development stages. The groups were 0 to 6 years old (infancy & early childhood), 6 to 12 years old (middle & late childhood), 12 to 18 years old (adolescence), 18 to 51 years old (early adulthood) and 51 to 84 years old (late adulthood). The number of autoantibodies in each subject was plotted using GraphPad Prism by age groups. To understand the effect of gender on autoantibody counts in healthy individuals, studies having both male and female subjects with matched age were used (Studies I, II, IV, VII, Table 2). The subjects were divided into male and female groups. The number of autoantibodies found in each subject was plotted using GraphPad Prism. The weighted prevalence of each autoantibody was calculated for male and female separately. The method of weighted prevalence calculation is described in the “Quantification and statistical analysis” subsection. Prevalence values for the 77 most common autoantibodies were plotted as a population pyramid using GraphPad Prism. A paired t-test was performed to determine the significance of the prevalence difference between genders. Pearson correlation of common autoantibodies frequency in diseased and healthy cohorts were plotted using Python seaborn package.

c. Correlation of Common Autoantibodies

As the presence of common autoantibodies were measured on a binary scale, a phi correlation coefficient was computed to measure associations between autoantibodies. Specifically, for each pair of antibodies, a phi correlation coefficient was computed for each study, and multiple phi correlation coefficients across different studies were combined into a single phi correlation coefficient using the R meta package. The R “pheatmap” package was then used to produce correlation heatmap plots for both healthy and diseased cohorts (FIGS. 8A and 8B). Here, phi correlation coefficient was not defined when one pair of antibodies showed no responses for all the samples, and these undefined phi correlation coefficients were colored as grey on the heatmap plots. Pairs of antibodies having correlation coefficient higher than 0.6 in both cohorts and have correlation in more than one study were validated.

d. Sequence Similarity with Viral Proteins

The proteomes of respiratory and common viruses found in children of the US were downloaded from UniProt as a FASTA file. All the common human viruses were included except sexually transmitted ones as common autoantibodies that develop early in age and then plateau (Table 4). CD-HIT was employed to remove duplicate sequences in the file (sequence identity cut-off: 1) (Huang et al., 2010). The sequences were then segregated into 14-mer peptides using a Python script (sliding window: 1) and consecutive amino acid repeats (3 or more) were removed. The sequences of all the human proteins analyzed on microarrays were retrieved from DNASU (https://dnasu.org) and split into two sequence databases, “common autoantigens” and “unreactive proteins”. The “unreactive proteins” database comprises proteins from the microarrays without any autoantibody responses. Repeats and low-complexity regions were masked using BLAST+(Basic Local Alignment Search Tool, version 2.10.1) package “segmasker”. A protein-protein BLAST was run with the following parameters, “-ungapped, -db_hard_mask 21, -comp_based_stats F, -evalue 10”, between viral 14-mer peptides and “common autoantigens”. Another protein-protein BLAST was run between viral 14-mer peptides and “unreactive proteins” with similar parameters except adjusted “-evalue 593.89” to compensate for the bigger size of unreactive proteins database (Effective search space of “unreactive proteins” and “common autoantigens” databases were 15,970,464 and 268,912, respectively). The total number of amino acids matches higher than the threshold (7 ungapped amino acids match) was calculated for both databases and compared with the total number of amino acids in each database using a chi-square test (FIG. 2A).

e. Biochemical and Structural Properties

Biopython (version 1.75) module Bio.SeqUtils.ProtParam for Python (version 3.7.6) was used to calculate the values of aromaticity, isoelectric point, hydrophobicity, the fraction of amino acids in sheets and turns for each protein (Table 8). Secondary structure and antigenicity prediction methods from Immune Epitope Database (IEDB) were also used. Command-line tools from IEDB analysis resource were employed to calculate the values of Chou & Fasman beta-turn, Emini surface accessibility, Karplus & Schulz flexibility, and Parker hydrophilicity across the proteins, which were then averaged for each protein. The computed biochemical property values were used for the enrichment analysis on the identified common autoantigens using Gene Set Enrichment Analysis (GSEA) “GSEAPreranked” package (version 4.2) (Subramanian et al., 2005).

f. Subcellular Localization and Tissue Expression

All 8,282 proteins were used to query the UniProt database for subcellular localization (downloaded in December 2020), among which 6,875 proteins had subcellular localization data available in the database (Table 6). Some of the proteins were found simultaneously in more than one location, and hence, seven groups were created to segregate the proteins based on their subcellular localization profiles. Proteins that were found only in one subcellular location were put into “intracellular only”, “cell membrane only” and “secreted only” groups. Proteins that were found in two subcellular locations were put into “intracellular & cell membrane”, “cell membrane & secreted” and “secreted & intracellular” groups. Proteins that were found simultaneously inside the cell, in the cell membrane, and outside the cell were put into “intracellular, cell membrane and secreted” group. P value was calculated to assess the statistical significance of difference in fractions of “intracellular only” proteins for all proteins on the microarrays and for common autoantigens using the proportion test.

All 8,282 proteins were mapped to the Ensembl IDs using “BiomaRt” package available for R (version 3.5.0). The Ensembl IDs were used to identify the protein of interest in the Genotype-Tissue Expression (GTEx, version 8) dataset. The gene expression levels in 52 human tissue types, measured in transcripts per million (TPM), were downloaded from GTEx. Expression values for tissue types belonging to the same organ were averaged. Differentially expressed genes for each organ/tissue were identified using edgeR package for R (version 3.6.2) with a cutoff of Log2 (fold change)>3 to determine organ/tissue specificity, where the fold change for each gene was calculated by dividing the TPM value in a particular organ/tissue by the mean TPM values in all other organs/tissues. The Log2-scaled fold changes across the organs/tissues for each gene were standardized to the Z scores for data visualization. The Z score profiles were displayed in a heatmap with correlation-based average-linkage clustering by using the Python seaborn package.

g. Quantification and Statistical Analysis

i. Weighted Prevalence

Due to the heterogeneous number of proteins and subjects being analyzed in each study, we computed the weight for the jth antibody as \hat{p}ⱼ=Σᵢ₌₁^{k}wᵢⱼpᵢⱼ/Σᵢ₌₁^{k}wᵢⱼ, where pᵢⱼ=xᵢⱼ/nᵢⱼ is the prevalence, xᵢⱼ is the total number of positive signals found for the jth antibody in the study i, and nᵢⱼ is the number of samples for the jth antibody in the study i, and k is the number of studies. Here, wᵢⱼ=(vᵢⱼ+τⱼ²)⁻¹ is the inverse variance-weighting which accounts for the heterogeneous effects between studies (Borenstein et al., 2010), where vᵢⱼ=nᵢⱼ/(pᵢⱼ(1−pᵢⱼ)), τⱼ²=(Qⱼ−k+1)/Uⱼ if Qⱼ>k−1 or τⱼ²=0 otherwise, Qⱼ=Σᵢ₌₁^{k}vᵢⱼ(pᵢⱼ−pⱼ)², Uⱼ=(k−1)(v̅ⱼ−sⱼ²/(kvⱼ)), pⱼ=Σᵢ₌₁^{k}vᵢⱼpᵢⱼ/Σᵢ₌₁^{k}vᵢⱼ, sⱼ²=(Σᵢ₌₁^{k}vᵢⱼ²−kv̅ⱼ²)/(k−1), and v̅ⱼ=Σᵢ₌₁^{k}vᵢⱼ/k. The same analysis was performed to calculate gender-specific weighted prevalence by splitting the dataset into male and female subsets.

ii. Age and gender comparison

The significance of increase in the autoantibody counts among the five age groups was calculated using the Welch's t-test while the significance of difference in autoantibody counts between the male and female groups was calculated using a two-sample unpaired t-test.

iii. Biochemical and structural properties

The “GSEAPreranked” package available in GSEA software returned P values of “0.0” when the number of permutations was set to 1000, as it cannot calculate very small P values. Another R package named “fgsea” was used to calculate the very small P values with number of permutations set to 10,000 for more accurate calculation. To adjust multiple comparisons, we computed false discovery rate (FDR) adjusted P value using the “p.adjust” function in the R stats package.

What is claimed is:

1. A method of increasing the accuracy of a cancer or autoimmune condition screening of a subject suspected of having cancer or an autoimmune condition by identifying at least one false positive indicator, the method comprising:
providing a biological sample from the subject;
detecting the presence of one or more autoantibodies against at least one autoantigen listed in Table 1 in the biological sample, and
identifying at least one false positive indicator upon detecting the presence of the one or more autoantibodies.

2. The method of claim 1, wherein:
the at least one autoantigen is selected from the group consisting of: STMN4, ODF2, RBPJ, AMY2A, EPCAM, ZNF688, CSF3, S1PR3, CDR2L, RASSF1, SPAG8, TRAP1, SART1, GATA2, PML, SOX2, PELI1, TAX1BP1, JUN, and PAK1;
the at least one autoantigen is selected from the group consisting of: AMY2A, SPAG8, GTSE1, MAK, RNF138, PMFBP1, CCDC34, ODF2, CSF3, CAPN3, MYLK2, TRIM29, SOX2, and STMN4; or
the at least one autoantigen is selected from the group consisting of: STMN4, ODF4, RBPJ, AMY2A, EPCAM, ZNF688, CSF3, RAD51AP1, PSKH1, LENG1, S1PR3, LYSMD1, FAM76A, CDR2L, and CCDC130.

3. The method of claim 2, wherein the biological sample is from the subject's pancreas, the at least one autoantigen is AMY2A.

4. The method of claim 2, wherein the biological sample is from the subject's lungs, the at least one autoantigen is CSF3.

5. The method of claim 2, wherein the biological sample is from the subject's salivary gland, nerve tibial, or brain, the at least one autoantigen is SOX2.

6. The method of claim 2, wherein the biological sample is from the subject's esophagus, skin, or vagina, the at least one autoantigen is TRIM29.

7. The method of claim 2, wherein the biological sample is from the subject's skeletal muscle, the at least one autoantigen is CAPN3 and/or MYLK2.

8. The method of claim 2, wherein the biological sample is from the subject's testis, the at least one autoantigen is selected from the group consisting of: SPAG8, GTSE1, MAK, RNF138, PMFBP1, CCDC34, and ODF2.

9. The method of claim 2, wherein the at least one autoantigen comprises EPCAM, the method further comprises detecting the presence of at least one other autoantibody against EDG3 or CSF3, wherein the presence of the at least one other autoantibody against EDG3 or CSF3 in the biological sample indicates the subject is false positive for having cancer or an autoimmune condition.

10. The method of claim 2, wherein the method comprises detecting at least two, at least three, at least four, or at least five autoantibodies against at least two, at least three, at least four, or at least five autoantigens selected from the group consisting of: STMN4, ODF4, RBPJ, AMY2A, EPCAM, ZNF688, CSF3, RAD51AP1, PSKH1, LENG1, S1PR3, LYSMD1, FAM76A, CDR2L, and CCDC130, the presence of the at least two, at least three, at least four, or at least five autoantibodies indicate the subject is false positive for having cancer or an autoimmune condition.

11. The method of claim 2, wherein the at least two, at least three, at least four, or at least five autoantibodies comprises EPCAM, the method further comprises detecting the presence of at least one other autoantibody against EDG3, wherein the presence of the at least one other autoantibody against EDG3 in the biological sample indicates the subject is false positive for having cancer or an autoimmune condition.

12. The method of claim 2, wherein the method comprises detecting at least ten autoantibodies against at least ten selected from the group consisting of: STMN4, ODF4, RBPJ, AMY2A, EPCAM, ZNF688, CSF3, RAD51AP1, PSKH1, LENG1, S1PR3, LYSMD1, FAM76A, CDR2L, and CCDC130, the presence of the at least ten autoantibodies indicate the subject is false positive for having cancer or an autoimmune condition.

13. The method of claim 2, wherein the method comprises detecting the presence of at least one autoantibody against STMN4, ODF4, RBPJ, AMY2A, EPCAM, ZNF688, CSF3, RAD51AP1, PSKH1, LENG1, S1PR3, LYSMD1, FAM76A, CDR2L, and CCDC130 in the biological sample, the presence of at least one autoantibody against STMN4, ODF4, RBPJ, AMY2A, EPCAM, ZNF688, CSF3, RAD51AP1, PSKH1, LENG1, S1PR3, LYSMD1, FAM76A, CDR2L, and CCDC130 in the biological sample indicate the subject is false positive for having cancer or an autoimmune condition.

14. The method of claim 2, wherein the method comprises detecting the presence of at least one autoantibody against at least one autoantigen selected from the group consisting of: STMN4, ODF2, RBPJ, AMY2A, EPCAM, and ZNF688 in the biological sample, wherein the presence of the at least one autoantibody indicates the subject is false positive for having cancer or an autoimmune condition.

15. The method of claim 14, wherein the at least one autoantigen comprises EPCAM, the method further comprises detecting the presence of at least one other autoantibody against EDG3 or CSF3, wherein the presence of the at least one other autoantibody against EDG3 or CSF3 in the biological sample indicates the subject is false positive for having cancer or an autoimmune condition.

16. The method of claim 14, wherein the method comprises detecting the presence of at least two, at least three, at least four, or at least five autoantibodies against at least two, at least three, at least four, or at least five autoantigens selected from the group consisting of: STMN4, ODF2, RBPJ, AMY2A, EPCAM, and ZNF688 in the biological sample, wherein the presence of the at least two, at least three, at least four, or at least five autoantibodies indicates the subject is false positive for having cancer or an autoimmune condition.

17. The method of claim 14, wherein the method comprises detecting the presence of at least one autoantibody against STMN4, ODF2, RBPJ, AMY2A, EPCAM, and ZNF688 in the biological sample, wherein the presence of the at least one autoantibody against STMN4, ODF2, RBPJ, AMY2A, EPCAM, and ZNF688 in the biological sample indicates the subject is false positive for having cancer or an autoimmune condition.

18. A method of verifying the accuracy of a diagnosis of a cancer or autoimmune condition in a subject, the method comprising:
providing a biological sample from the subject;
detecting the presence of one or more autoantibodies against at least one autoantigen listed in Table 1 in the biological sample, and
identifying the subject as falsely diagnosed with cancer or an autoimmune condition upon detecting the presence of the one or more autoantibodies.

19. The method of claim 14, wherein
the at least one autoantigen is selected from the group consisting of: STMN4, ODF2, RBPJ, AMY2A, EPCAM, ZNF688, CSF3, S1PR3, CDR2L, RASSF1, SPAG8, TRAP1, SART1, GATA2, PML, SOX2, PELI1, TAX1BP1, JUN, and PAK1;
the at least one autoantigen is selected from the group consisting of: AMY2A, SPAG8, GTSE1, MAK, RNF138, PMFBP1, CCDC34, ODF2, CSF3, CAPN3, MYLK2, TRIM29, SOX2, and STMN4; or
the at least one autoantigen is selected from the group consisting of: STMN4, ODF4, RBPJ, AMY2A, EPCAM, ZNF688, CSF3, RAD51AP1, PSKH1, LENG1, S1PR3, LYSMD1, FAM76A, CDR2L, and CCDC130.

20. A kit for diagnosing and verifying an autoimmune condition or cancer in a subject, the kit comprising:
at least one antigen or antibody targeting a cancer marker, an autoimmune marker, or both; and
a false positive indicator, the false positive indicator comprising at least one autoantigen listed in Table 1;
at least one labeled secondary antibody or antigen for detecting the binding of the at least one antigen or antibody with the cancer marker and/or the autoimmune marker; and
at least one labeled secondary antibody or antigen detecting binding of at least one autoantibody to the false positive indicator.


SEQUENCE LISTING (34 sequences)

SEQ ID NO: 1 - protein, 7 aa, synthetic construct
SYFGLRT

SEQ ID NO: 2 - protein, 7 aa, synthetic construct
LRQEINA

SEQ ID NO: 3 - protein, 7 aa, synthetic construct
WPEGYQL

SEQ ID NO: 4 - protein, 7 aa, synthetic construct
ARCETQN

SEQ ID NO: 5 - protein, 7 aa, synthetic construct
LPVPPGG

SEQ ID NO: 6 - protein, 7 aa, synthetic construct
GKLNVKL

SEQ ID NO: 7 - protein, 7 aa, synthetic construct
SAGTSST

SEQ ID NO: 8 - protein, 7 aa, synthetic construct
NRSGYSG

SEQ ID NO: 9 - protein, 7 aa, synthetic construct
ALLAAGS

SEQ ID NO: 10 - protein, 7 aa, synthetic construct
EDCLYEL

SEQ ID NO: 11 - protein, 7 aa, synthetic construct
EQLRAKL

SEQ ID NO: 12 - protein, 7 aa, synthetic construct
AKLNREE

SEQ ID NO: 13 - protein, 7 aa, synthetic construct
SSNTVVA

SEQ ID NO: 14 - protein, 7 aa, synthetic construct
TIKRTLV

SEQ ID NO: 15 - protein, 7 aa, synthetic construct
ARCETQN

SEQ ID NO: 16 - protein, 7 aa, synthetic construct
GTSEVDE

SEQ ID NO: 17 - protein, 7 aa, synthetic construct
WPEGYQL

SEQ ID NO: 18 - protein, 7 aa, synthetic construct
KSDSELS

SEQ ID NO: 19 - protein, 7 aa, synthetic construct
VIGLLDV

SEQ ID NO: 20 - protein, 7 aa, synthetic construct
IDQNTVV

SEQ ID NO: 21 - protein, 7 aa, synthetic construct
SPIKLSP

SEQ ID NO: 22 - protein, 8 aa, synthetic construct
TAEEGKNI

SEQ ID NO: 23 - protein, 8 aa, synthetic construct
KAEEGKNI

SEQ ID NO: 24 - protein, 7 aa, synthetic construct
SVIEPLP

SEQ ID NO: 25 - protein, 8 aa, synthetic construct
NATAQELL

SEQ ID NO: 26 - protein, 8 aa, synthetic construct
NATAQELL

SEQ ID NO: 27 - protein, 7 aa, synthetic construct
TLGAVVP

SEQ ID NO: 28 - protein, 8 aa, synthetic construct
SPRKQEAE

SEQ ID NO: 29 - protein, 7 aa, synthetic construct
ELTVAAR

SEQ ID NO: 30 - protein, 7 aa, synthetic construct
DATHLED

SEQ ID NO: 31 - protein, 8 aa, synthetic construct
ALIRKLRQ

SEQ ID NO: 32 - protein, 8 aa, synthetic construct
ALIRKLRD

SEQ ID NO: 33 - protein, 7 aa, synthetic construct
AQLGPRR

SEQ ID NO: 34 - protein, 7 aa, synthetic construct
GAQPPAP